SEQUENCE LISTING (9 sequences)

SEQ ID NO: 1 - DNA, 1182 bp, Artificial Sequence
  PhaA gene
atgactgacgtggtgattgtttctgctgctagaacggctgttggaaagtttggtggatccttagctaaaattcccgcccctgaacttggtgctgtagtcataaaggcagcgttagagagagctggtgtgaaaccagaacaagtctctgaagtaatcatgggtcaagttctgacagctggttctggccaaaatccagcaagacaagcagccataaaggctggtttgcctgctatggtaccagcaatgaccattaacaaagtgtgtggcagcggtttgaaagcagtcatgctagctgctaacgcaattatggctggagatgcagagattgttgtggcaggagggcaagaaaatatgtcagccgcacctcatgttttgccaggttcaagagatggattcagaatgggtgatgctaagttggtagacaccatgatcgttgatggtctttgggacgtatacaaccagtaccatatggggattacagccgaaaacgttgctaaggagtatggcataactagagaagcgcaggatgaattggctgttggtagtcagaataaggcggaagctgcacagaaagctggcaagtttgatgaggaaattgtcccggtcttgataccccaaagaaaaggcgatcctgttgcctttaagacggatgaattcgttcgtcaaggggccactctagactcgatgagtggactgaaaccagccttcgataaagctggcacagtcactgctgcaaatgcatcaggtctaaatgatggcgcagcagcagttgttgtgatgagtgctgcgaaagccaaagaattggggttaacaccgctagccaccataaagtcctatgccaatgccggagttgaccccaaagttatgggcatgggccctgtaccagcctctaaaagggcgttatcgagagcggaatggacacctcaagacttagaccttatggagatcaatgaggcatttgcggctcaagctttggcagtacaccagcaaatgggttgggatacttccaaagtcaacgtaaatggaggtgcaatagccattgggcatccaatcggtgcaagcggttgtaggatccttgtcactctgttacacgaaatgaaacgtagggatgctaagaaaggtttagcctcattgtgcattggtggaggtatgggtgtggctttagccgttgaaagaaagtaa

SEQ ID NO: 2 - DNA, 741 bp, Artificial Sequence
  PhaB gene
atgacgcaaaggattgcgtatgtcacaggtggtatgggtggaataggtactgctatttgccaaagattagcgaaagacggttttagggtagttgccggttgtggtcccaacagtccaagaagggaaagatggttggaacaacagaaggcactaggttttgacttcgttgcatctgagggtaatgttgcagattgggactcaacgaaaacagcctttgacaaggtgaaagcggaagtaggagaagtcgatgtactgatcaacaatgcaggcataacaagagatgtcgttttccgtaagatgactagagctgattgggatgctgttattgacaccaatttgacgagcttgttcaacgttaccaagcaagtgattgatggcatggctgatagaggttggggtagaatcgtgaatatttcctcagttaacggacagaaaggccaatttggtcaaaccaactattctacagctaaagcagggttacatggctttaccatggctttagctcaagaagtggccactaaaggggtgactgtcaatacagtttctccaggatacattgctactgatatggtaaaagccatcagacaggatgtccttgacaagatcgttggcactattccggtaaagcgtctaggggaacctgaagagatagcctccatatgtgcttggctttcgtctgatgagtcaggatttagtacaggagcagatttcagcttgaatggtggcttacacatgggttaa

SEQ ID NO: 3 - DNA, 1770 bp, Artificial Sequence
  PhaC gene
atggccactggtaaaggggctgccgctagtacgcaagaaggcaaatcccaaccgtttaaatttacaccagggccttttgaccccgccacttggttagaatggtctagacaatggcaaggtacagaaggaaatggtcatgctgctgcttccggtatacccggtctagatgctttagctggtgtcaagattgaaccggcacaactaggtgatatacagcagagatacatgaaggacttttctgcactttggcaagctatggctgaagggaaagcagaagcaacaggccctcttcatgataggagattcgccggagatgcgtggaggacgaatttgccatataggttcgccgcagcgttttacctgttaaatgctagagcattgactgagttggcagatgctgtagaagctgacgctaaaacgagacaaagaattcgttttgccatttctcagtgggtcgacgctatgtcaccagctaattttctggcgacaaatcccgaggcacaaagactattaattgagtctggtggcgagtcgttaagagccggtgttaggaatatgatggaagatttgaccagaggcaagatatctcaaactgacgaatctgcgtttgaagtcggtagaaacgtagctgtctcagaaggagcggtcgttttcgagaatgagtatttccagttgttacaatataagccactgaccgacaaagtgcatgctagaccacttctaatggttcctccgtgcataaacaagtactatatcttagatttgcaacctgaatcttccttagttcgtcacgtagttgaacaaggtcatacagtctttctagtttcctggagaaatccggatgcatcgatggctggtagcacttgggacgattacatcgaacatgctgccattcgtgccattgaagtagcgagagatatctcaggccaagataaaatcaacgttttgggcttttgtgtaggtggtaccattgtttcaacagcattggctgtcatggcagctagaggtcaacatccagcggcaagtgttactttgttaacgactttactggatttcgctgataccggcatcttagatgtgttcgtcgatgaaggtcacgttcagcttcgtgaagctacattaggaggagctgctggagcaccatgtgctttgcttagagggctagagctagcaaacacttttagcttcttgaggccaaatgacttggtttggaattacgtagtggacaattatttgaaaggtaacacaccagttcccttcgatcttttgttttggaacggagatgccaccaatctaccaggtccatggtattgctggtacttgagacatacctatctgcaggatgaactgaaagttcctgggaagttgacagtatgtggtgttccagttgacttggcaagtatcgatgtacctacttacatatatggatcgagagaagatcacatagtgccttggactgcagcctatgcttcaacggcgttacttgccaacaagttaagattcgtgttaggggcatccggtcacattgcaggcgtgattaaccctcccgccaagaacaaacgtagccattggaccaatgatgctttgccagaatcaccacagcaatggttagccggagcaactgagcatcacggcagttggtggcctgactggacagcttggttggccggtcaagccggtgccaaaagagcagcccctgcaaattacggaaacgctagatatcccgctattgagccagcgcctggtaggtatgtgaaagcaaaggcataa

SEQ ID NO: 4 - DNA, 30 bp, Artificial Sequence
  PhaA forward primer
ggcaagaaaatatgtcagccgcacctcatg

SEQ ID NO: 5 - DNA, 30 bp, Artificial Sequence
  PhaA reverse primer
ggcataggactttatggtggctagcggtgt

SEQ ID NO: 6 - DNA, 30 bp, Artificial Sequence
  PhaB forward primer
aagggaaagatggttggaacaacagaaggc

SEQ ID NO: 7 - DNA, 35 bp, Artificial Sequence
  PhaB reverse primer
gaatagtgccaacgatcttgtcaaggacatcctgt

SEQ ID NO: 8 - DNA, 35 bp, Artificial Sequence
  PhaC forward primer
tctagatgctttagctggtgtcaagattgaaccgg

SEQ ID NO: 9 - DNA, 30 bp, Artificial Sequence
  PhaC reverse primer
cattctcgaaaacgaccgctccttctgaga

(19) United States
(12) Patent Application Publication
(10) Pub. No.: US 2023/0235370 A1
(43) Pub. Date: Jul. 27, 2023

(54) BIOSYNTHESIS OF POLYHYDROXYBUTYRATE

(71) Applicant: Trade Wind Biotech Co., Ltd, Taipei City (TW)

(72) Inventor: JUI-JEN CHANG, Taipei City (TW)

(21) Appl. No.: 17/583,207

(22) Filed: Jan. 25, 2022

(51) Int. Cl. C12P 7/6463; C12N 15/52; C12N 1/16; C12N 9/10

(52) U.S. Cl. CPC C12P 7/6463; C12N 15/52; C12N 1/16; C12N 9/1029

(57) ABSTRACT

A method for biosynthesis polyhydroxybutyrate by a yeast transformant of the invention includes the following steps: (1) transforming a polyhydroxybutyrate biosynthesis related gene into an oleaginous yeast to obtain an yeast transformant. (2) screening the yeast transformant. (3) cultivating the yeast transformant to obtain the polyhydroxybutyrate. The method of the invention provides a way of cheaper, faster and flexibility in biotechnology metabolism to improve PHB production.

### REFERENCE TO SEQUENCE LISTING

This application contains a sequence listing submitted as an ASCII text file, named “Sequence-Listing.txt” and created on Jan. 20, 2022, with 7 kilobytes in size. The material in the above-identified ASCII text file is incorporated herein by reference in its entirety.

### FIELD OF THE INVENTION

The present invention relates to a method for biosynthesis polyhydroxybutyrate, and more particularly to a method for biosynthesis polyhydroxybutyrate by a yeast transformant.

### BACKGROUND OF THE INVENTION

Unsustainability degradation of petroleum plastic threatens the stability of ecosystem on land and marine. Increasing plastic consumption in the world encourages the transformation of raw materials plastic to become biodegradable plastic. The most common type of biodegradable polymers of plastics is polyhydroxyalkanoates (PHAs). Polyhydroxybutyrate (PHB) is one of the most studied PHAs. Both of them were the best examples of biopolymer energy storage and having similar biosynthesis properties to synthetic biopolymers.

Since, PHB was discovered by Lemoigne in 1926 in Bacillus megaterium as cytoplasmic inclusion. It has attracted much industrial attention as a biodegradable and biocompatible thermoplastic. Moreover, it can be generated using renewable and non-renewable resources to obtain biodegradable polymers. PHB is the most important example of a biocompatible and bio-degradable hydrophobic polymer at high melting temperatures and crystallinity.

Some tremendous potential applications of PHB are used in biomedical, pharmacological, waste management, veterinary, agricultural, and novel biofuel. Some microorganisms successfully produce PHB by natural products from wild-type or recombinant organisms, such as bacterial, cyanobacteria and green algae. In controlled aerobic and anaerobic cultivation, about 300 species of bacteria and archaea have been discovered to produce PHA and PHB.

Some technologies have been improved PHB content from microbial cell factories from both wildtype and recombinant, such as Cupriavidus necator, Bacillus sp., Synechocystis sp., Recombinant E. coli and Halomonas sp. Nevertheless, the cost production in bacterial technologies was very expensive and involving many complicated methods.

The information disclosed in this “BACKGROUND OF THE INVENTION” section is only for enhancement understanding of the background of the invention and therefore it may contain information that does not form the prior art that is already known to a person of ordinary skill in the art. Furthermore, the information disclosed in this “BACKGROUND OF THE INVENTION” section does not mean that one or more problems to be solved by one or more embodiments of the invention were acknowledged by a person of ordinary skill in the art.

### SUMMARY OF THE INVENTION

The present invention provides a method for biosynthesis polyhydroxybutyrate by a yeast transformant, which provides a way of cheaper, faster and flexibility in biotechnology metabolism to improve PHB production.

Other advantages and objects of the invention may be further illustrated by the technical features broadly embodied and described as follows.

In order to achieve one or a portion of or all of the objects or other objects, a method for biosynthesis polyhydroxybutyrate by a yeast transformant provided in an embodiment of the invention includes the following steps: (1) transforming a polyhydroxybutyrate biosynthesis related gene into an oleaginous yeast to obtain an yeast transformant. (2) screening the yeast transformant. (3) cultivating the yeast transformant to obtain the polyhydroxybutyrate.

In one embodiment of the invention, the oleaginous yeast is Rhodotorula glutinis.

In one embodiment of the invention, a Rhodotorula glutinis strain is BCRC 22360.

In one embodiment of the invention, the polyhydroxybutyrate biosynthesis related gene comprises at least one of PhaA gene, PhaB gene or PhaC gene.

In one embodiment of the invention, the polyhydroxybutyrate biosynthesis related gene is PhaA gene, PhaB gene and PhaC gene.

In one embodiment of the invention, the polyhydroxybutyrate biosynthesis related gene comprises at least one of a first gene having at least 90% sequence identity with a sequence of PhaA gene, a second gene having at least 90% sequence identity with a sequence of PhaB gene or a third gene having at least 90% sequence identity with a sequence of PhaC gene.

In one embodiment of the invention, the method of transforming a polyhydroxybutyrate biosynthesis related gene into an oleaginous yeast to obtain the yeast transformant comprises inserting the polyhydroxybutyrate biosynthesis related gene into a linearized plasmid, and transforming the linearized plasmid into the oleaginous yeast.

In one embodiment of the invention, a DNA of the yeast transformant has the polyhydroxybutyrate biosynthesis related gene.

In one embodiment of the invention, the method of screening the yeast transformant comprises screening with a primer pair, one of the primer of the primer pair has a sequence as SEQ ID NO: 4, and the other primer has a sequence as SEQ ID NO: 5.

In one embodiment of the invention, the method of screening the yeast transformant comprises screening with a primer pair, one of the primer of the primer pair has a sequence as SEQ ID NO: 6, and the other primer has a sequence as SEQ ID NO: 7.

In one embodiment of the invention, the method of screening the yeast transformant comprises screening with a primer pair, one of the primer of the primer pair has a sequence as SEQ ID NO: 8, and the other primer has a sequence as SEQ ID NO: 9.

In one embodiment of the invention, the method of cultivating the yeast transformant to obtain the polyhydroxybutyrate comprises cultivating under aerobic condition.

In one embodiment of the invention, the method of cultivating the yeast transformant to obtain the polyhydroxybutyrate comprises cultivating the yeast transformant with oil.

In one embodiment of the invention, a yield of polyhydroxybutyrate increase when cultivating the yeast transformant with glucose.

In one embodiment of the invention, a production capacity of polyhydroxybutyrate per cell increase when cultivating the yeast transformant with glycerol or oil.

In one embodiment of the invention, the method of cultivating the yeast transformant to obtain the polyhydroxybutyrate comprises cultivating the yeast transformant in seawater.

Since yeast has flexibility in physiology, is novel in biotechnology metabolism and larger than bacteria, it provides a cheaper and faster way to improve PHB production. In the method of the embodiment of the invention, an oleaginous yeast is used in PHB production, which belongs to a type of yeast that has ability to provide lipids as a substrate for PHB synthesis in metabolic plasticity. It is demonstrated that an increasing of PHB production in oleaginous yeast by using optimation some carbon sources. Therefore, comparing with the method of using bacteria in the conventional technology, the oleaginous yeast of the embodiment of the invention provides a way of cheaper, faster and flexibility in biotechnology metabolism, and may further increase the PHB production.

Other objectives, features and advantages of The invention will be further understood from the further technological features disclosed by the embodiments of The invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic flow diagram of a method for biosynthesis polyhydroxybutyrate by a yeast transformant of one embodiment of the invention;

FIG. 2 is a schematic diagram of PHB pathway;

FIG. 3 is total colonies of candidate transformants Rhodotorula glutinis in different variety minimal inhabitation concentration of antibiotic G418. (3A) Wild-type. (3B) Colony transformant Rhodotorula glutinis on YPAD+5 μg/ml G418. (3C) Colony transformant Rhodotorula glutinis on YPAD+10 μg/ml G418. (3D) Colony transformant Rhodotorula glutinis on YPAD+25 μg/ml G418. (3E) Colony transformant Rhodotorula glutinis on YPAD+50 μg/ml G418. (3F-3G) Colony transformant Rhodotorula glutinis on YPAD +100 μg/ml G418. (3H-3I) Colony transformant Rhodotorula glutinis on YPAD+200 μg/ml G418;

FIG. 4 is randomly selected and sub-cultured of a single colony of candidate transformants Rhodotorula glutinis in different growth variety concentrations of antibiotic G418. (4A) Single colony transformant Rhodotorula glutinis on YPAD+5 μg/ml G418. (4B) Single colony transformant Rhodotorula glutinis on YPAD+10 μg/ml G418. (4C) Single colony transformant Rhodotorula glutinis on YPAD+25 μg/ml G418. (4D) Single colony transformant Rhodotorula glutinis on YPAD+50 μg/ml G418. (4E) Single colony transformant Rhodotorula glutinis on YPAD+100 μg/ml G418;

FIG. 5 is PCR validation for three genes (PhaA-1200 bp, PhaB- 800 bp and PhaC-1800 bp) in Rhodotorula glutinis candidate transformants. The numbers represent: 1: 25-39; 2: 25-44, 3: 50-39, 4: 50-87, 5: 100-12, 6:100-13, 7: 100-15, 8: 100-16, 9: 100-29, 10: Wildtype;

FIG. 6 is western blot analysis of three enzymes PhaA (˜66 KDa), PhaB (44 KDa) and PhaC (29 KDa) of PHB biosynthesis in Rhodotorula glutinis transformants. 1: 25-39; 2: 25-44, 3: 50-39, 4: 50-87, 5: 100-12, 6:100-13, 7: 100-15, 8: 100-16, 9: 100-29, 10: Wild-type;

FIGS. 7A-7K are HPLC analysis of PHB accumulation in nine selected Rhodotorula glutinis transformants. FIG. 7A PHB standard peak for 0.25 g/L. FIG. 7B Strain 25-39. FIG. 7C Strain 25-44. FIG. 7D Strain 50-39. FIG. 7E Strain 50-87. FIG. 7F Strain 100-12. FIG. 7G Strain 100-13. FIG. 7H Strain 100-15. FIG. 7I Strain 100-16. FIG. 7J Strain 100-29. FIG. 7K Wildtype. The peak at 6 min represents to the HPLC front;

FIGS. 8A-8B are performance of nine selected Rhodotorula glutinis transformants for the production of PHB. FIG. 8A PHB concentration, FIG. 8B PHB yield and PHB content (n=3);

FIGS. 9A-9D are performance of engineered Rhodotorula glutinis strain #100-29 in the shaken cultures using glucose, galactose, crude glycerol and oil each 30 g/L. FIG. 9A pH, biomass and PHB concentration. FIG. 9B PHB yield and PHB content. FIG. 9C Lipid content and total lipid. FIG. 9D The residue of carbon sources. n=3 replicates;

FIGS. 10A-10D are performance of engineered Rhodotorula glutinis strain #100-29 in the shaken cultures using different concentrations of glucose. FIG. 10A pH, biomass and PHB concentration. FIG. 10B PHB yield and PHB content. FIG. 10C Lipid content and total lipid. FIG. 10D The residue of glucose. n=3 replicates;

FIGS. 11A-11D are performance of engineered Rhodotorula glutinis strain #100-29 in the batch fermentation using 30 g/L glucose and crude glycerol as carbon sources. FIG. 11A Biomass and PHB concentration. FIG. 11B PHB yield and PHB content FIG. 11C Total lipid and lipid content. FIG. 11D Residue of glucose and glycerol. n=3 replicates; and

FIGS. 12A-12D are performance of engineered Rhodotorula glutinis strain #100-29 in the batch fermentation using different concentrations ofcrude glycerol. FIG. 12A Biomass. FIG. 12B PHB concentration. FIG. 12C PHB yield. FIG. 12D PHB content. n=3 replicates.

FIGS. 13A-13C are performance of engineered R. glutinis strain #100-29 in the shaken cultures using different concentrations of NaCl. FIG. 13A Biomass and PHB concentration. FIG. 13B PHB yield and PHB content. FIG. 13C The residue of carbon sources. n=3 replicates.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

Unless otherwise specified, all technical and scientific terms used herein have the same meaning as commonly understood by those with ordinary knowledge in the technical field to which the present invention belongs.

The article “a”, “an” and “the” used here refers to one or more (ie, at least one) grammatical acceptors of the article.

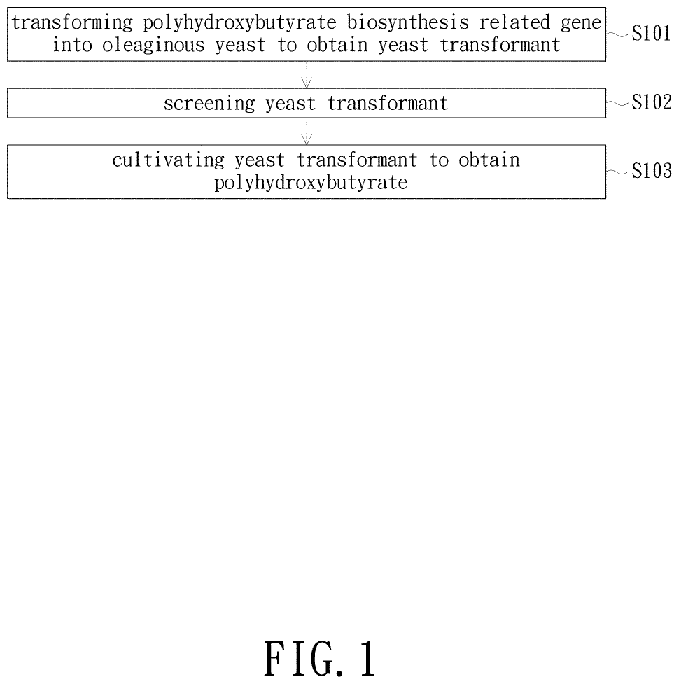

FIG. 1 is a schematic flow diagram of a method for biosynthesis polyhydroxybutyrate by a yeast transformant of one embodiment of the invention. Referring to FIG. 1, a method for biosynthesis polyhydroxybutyrate by a yeast transformant provided includes the following steps. Step S101: transforming a polyhydroxybutyrate (hereinafter “PHB”) biosynthesis related gene into an oleaginous yeast to obtain an yeast transformant. Oleaginous yeasts have a unique physiology that makes them the best suited hosts for the production of lipids, oleochemicals, and diesel-like fuels. Their high lipogenesis, capability of growing on many different carbon sources (including lignocellulosic sugars), easy large-scale cultivation, and an increasing number of genetic tools are some of the advantages that have encouraged their use to develop sustainable processes. In a preferred embodiment, the oleaginous yeast is Rhodotorula glutinis. Rhodotorula glutinis is a member of oleaginous yeast that has the capability to produce PHB, due to its ability to provide lipids as a substrate for PHAs synthesis in metabolic plasticity. A Rhodotorula glutinis strain is BCRC 22360. A plasmid strain used in the transformation is JM 109, and pKLAC2-na- (Biolabs, Inc.) as a plasmid for construct genes cassettes.

In the embodiment, the basic growth media for plasmid transformant contained 25 g/L LB broth with antibiotics (for example, Ampicillin). Rhodotorula glutinis are cultured in YP2D at 24° C. at 250 rpm several times, as follows: 1% yeast extract, 1% peptone and 20% glucose. The medium for transformant Rhodotorula glutinis is 1% yeast extract, 2% peptone, 20% galactose, and antibiotics (G418). The medium for seed screening and seed culture of transformants Rhodotorula glutinis are provided on a defined medium (21 g/L YM broth) in a-250 flasks using 50 ml as working volume. The fermentation medium (per liter) (in shaking flask cultivation and aerobic batch bioreactor) is comprised of 2 g of yeast extract, 2 g of (NH₄)₂SO₄, 1 g of KH₂PO₄, 0.5 g of MgSO₄·7H₂O, 0.1 g of CaCl₂ and 0.1 g of NaCl, which is expressed as the standard medium in the embodiment. Carbon sources mix in fermentation media when in shaking flask cultivation comprised of glucose (30 g/L; 45 g/L and 60 g/L), galactose (30 g/L), crude glycerol (30 g/L) from biodiesel industry and WCO (30 g/L). Carbon sources mix in aerobic batch fermentation media using glucose and crude glycerol in similar concentrations (30 g/L). In the crude glycerol experiment, carbon sources mix in aerobic batch fermentation media using glucose (30 g/L) and crude glycerol (30 g/L; 60 g/L and 90 g/L). In the NaCl experiment, the fermentation medium (per liter) in the aerobic batch bioreactor was comprised of 2 g of yeast extract, 2 g of (NH₄)₂SO₄, 1 g of KH₂PO₄, 0.5 g of MgSO₄·7H₂O, 0.1 g of CaCl₂ and (0.1 g as control; 1 g; 2 g; 3 g; 4 g and 5 g) of NaCl. Carbon sources mixed in aerobic batch fermentation media using glucose and crude glycerol in similar concentrations (30 g/L).

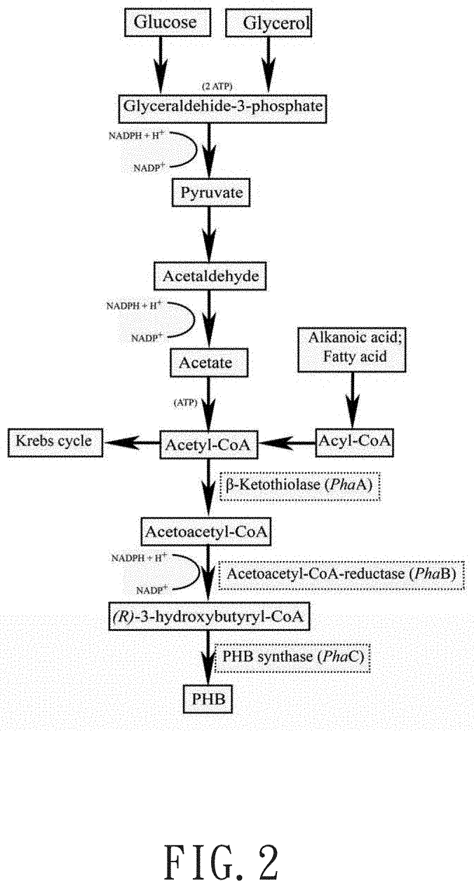

The biosynthesis pathway of PHB involves three enzymes and there in order essential reactions including acetyl-CoA C-acetyltransferase, NADPH-dependent acetoacetyl-CoA reductase, and PHA synthase as shown in FIG. 2. Synthetic biology and the genomic toolkit of yeast are complete and aided by contemporary techniques. The aims of the invention are three folds. First, intensively examined synthetic biology tools to improve the PHB production through transforming PHB biosynthesis related genes into Rhodotorula glutinis genome. Second, screened the best candidates of transformant Rhodotorula glutinis by western blot analysis and HPLC method and measurement PHB productivity. Third, demonstrated increasing the productivity of PHB in selected the best engineered Rhodotorula glutinis through fermentation both shaking flask cultivation and aerobic batch bioreactor using optimation some carbon sources.

In the embodiment, the polyhydroxybutyrate biosynthesis related gene comprises at least one of PhaA gene (GenBank: KP681582, SEQ ID NO: 1), PhaB gene (GenBank: KP681583, SEQ ID NO: 2) or PhaC gene (GenBank: KP681584, SEQ ID NO: 3). That is, the PHB biosynthesis related gene of the invention may be PhaA gene and genes other than PhaB gene and PhaC gene, PhaB gene and genes other than PhaA gene and PhaC gene, PhaC gene and genes other than PhaA gene and PhaB gene, PhaA gene and PhaB gene and genes other than PhaC gene, PhaB gene and PhaC gene and genes other than PhaA gene, PhaA gene and PhaC gene and genes other than PhaB gene, and PhaA gene, PhaB gene and PhaC gene. In another preferred embodiment, the PHB biosynthesis related gene is PhaA gene, PhaB gene and PhaC gene.

In the biosynthesis pathway of PHB, the acetyl-CoA C-acetyltransferase is encoded by PhaA gene or a first gene having at least 90% sequence identity with a sequence of PhaA gene. The NADPH-dependent acetoacetyl-CoA reductase is encoded by PhaB gene of a second gene having at least 90% sequence identity with a sequence of PhaB gene. The PHA synthase is encoded by PhaC gene or a third gene having at least 90% sequence identity with a sequence of PhaC gene. Therefore, the PHB biosynthesis related gene may also be regarded as comprising at least one of the first gene, the second gene or the third gene.

In the embodiment, the PHB biosynthesis related gene is PhaA gene, PhaB gene and PhaC gene is taken as an example, but not limited thereto. The PhaA gene, the PhaB gene, and the PhaC gene from Cupriavidus necator were codon-optimized towards S. cerevisiae and synthesized (TWIST BIOSCIENCE, USA). Sequence design & optimization for three PHB pathway constructions (pKLAC2-na-PhaA, pKLAC2-na-PhaB, pKLAC2-na-PhaC) are inserted in the plasmid (pKLAC2-na-).

To transform the PHB biosynthesis related gene into the oleaginous yeast Rhodotorula glutinis, the PHB biosynthesis related gene is first inserted into a linearized plasmid pKLAC2-na- (SacII digestion). Then, Rhodotorula glutinis strain BCRC 22360 are transformed with the linearized plasmid pKLAC2-na- according to Pi H W, Anandharaj M, Kao Y Y, et al. (2018) Engineering the oleaginous red yeast Rhodotorula glutinis for simultaneous β-carotene and cellulase production. Sci Rep 8:2-11. After the transformation, the linearized plasmid is pasted to a DNA of the oleaginous yeast Rhodotorula glutinis without being existed independently in the body of the oleaginous yeast, that is, a DNA of the yeast transformant has the PHB biosynthesis related gene. The advantage of pasting the linearized plasmid to the DNA of the oleaginous yeast Rhodotorula glutinis is when cultivating the yeast transformant, the PHB biosynthesis related gene may follow the DNA replication of the oleaginous yeast more stably and reduce the occurrence of errors.

The Rhodotorula glutinis are cultivated in 5 ml YP2D from the single colony at 30° C. at 250 rpm and then 0.2 OD cells are sub-cultured into 50 ml YP2D until reaching 0.6˜1.4 OD. Cells are harvested at 3000 rpm for 3 min (4° C.) and washed with 5 ml ice-cold distilled H₂O two times. Then, cells are resuspended in cooled TMLSD buffer (10 mM Tris-HCl buffer (pH 8.0) containing 2 mM MgCl₂, 100 mM lithium acetate, 270 mM sucrose, 10 mM dithiothreitol) and incubated at 24° C. for 1 h with shaking for 250 rpm. After the incubation, cells are harvested as described above and first washed using with 5 ml ice-cold distilled H₂O and second with 1 ml TMS buffer (10 mM Tris-HCl buffer (pH 8.0) containing 2 mM MgCl₂, 270 mM sucrose). Finally, competent cells are resuspended in 250 μL TMS buffer and prepared for electroporation analysis.

Electroporation procedure referred to Pi H W, Anandharaj M, Kao Y Y, et al. (2018) Engineering the oleaginous red yeast Rhodotorula glutinis for simultaneous β-carotene and cellulase production. Sci Rep 8:2-11, by mixing the 10-15 μl DNA with 40 μl competent cells and kept on ice for 15 min. Then cells are transferred to the ice-cold aluminum cuvette (0.2 cm gap Gene Pulser/MicroPulser Electroporation Cuvettes, Bio-Rad, USA) and electroporation is performed (1000 V, 400 Ω, and 25 μF capacitance), using a MicroPulser (Bio-Rad Laboratories, USA). After electroporation, cells are resuspended in 1 mL ice-cold YP2D and transferred into new tubes on ice for 15 min, and then incubated at 30° C. for 12 h. The cell suspension is spread onto YP2D plates containing selection markers (G418) and incubated at 30° C. for 4-5 days.

Next, Step S102: screening the yeast transformant. The successfully engineered colonies are validated by PCR amplification of the integrated gene, using extracted genomic DNA as a template. More than 380 single colonies are confirmed by PCR using gene-specific primer pairs. For example, screening the PhaA gene with a primer pair, one of the forward primer of the primer pair has a sequence as SEQ ID NO: 4, and the other reverse primer has a sequence as SEQ ID NO: 5, but not limited thereto. For example, screening the PhaB gene with a primer pair, one of the forward primer of the primer pair has a sequence as SEQ ID NO: 6, and the other reverse primer has a sequence as SEQ ID NO: 7, but not limited thereto. For example, screening the PhaC gene with a primer pair, one of the forward primer of the primer pair has a sequence as SEQ ID NO: 8, and the other reverse primer has a sequence as SEQ ID NO: 9, but not limited thereto.

Western Blot Analysis

Yeast protein extraction was determined by standard method procedure from a company (Bio Basic). Briefly, ten candidate transformant Rhodotorula glutinis are cultivated in YM broth at 24° C. until OD600 of yeast cell density reaches ˜1.0 or so. The culture cell is centrifugated at 8000 rpm for one minute, then discard supernatant and washed with sterilized water and kept yeast paste. Subsequently, yeast paster is added 500 μl isosmotic buffer, 5 μl Snailase buffer and 1 μl mercaptoethanol per 50 mg wet yeast paste. Then mixs gently with up and down solution and yeast paste to fully re-suspend yeast cells. Incubate at 37° C. for 2 hours and occasionally invert the tube for several times. Subsequently, centrifugate at 5000 rpm for one minute and discard supernatant and kept precipitates. Precipitates are washed with 500 μl isosmotic buffer, then centrifugate at 5000 rpm for one minute, and kept protoplasmic precipitates. This step could repeat once more. In the last step, 500 μl hypoosmotic buffer and 5 μl PMSF solution are added in protoplasmic precipitates and then vortex to fully re-suspend protoplasmic. The lysed solutions are kept at −20° C. for 30 minutes, and then thaw at room temperature. This step could repeat once more and store at −20° C. for further western blot.

Western blot analysis is performed using Lee M H, Hsu T L, Lin J J, et al. (2020) Constructing a human complex type N-linked glycosylation pathway in Kluyveromyces marxianus. PLoS One 15:1-16. Firstly, for measuring total protein concentration, the lysate of protein is mixed with bicinchoninic acid from Pierce™ BCA Protein Assay Kit. Then, the concentration of protein is measured using Sunrise, Tecan apparatus completely with Elisa reader. Subsequently, 10-50 μg of lysate is loaded to Tris-glycine SDS-Polyacrylamide Gel Electrophoresis using a combination of the Resolving gels (bottom gel) and 5% of the Stacking gels (upper gel). The Resolving gels are comprised of 30% Acrylamide mix, 1.5M Tris (pH 8.8), 10% SDS, 10% Ammonium Persulfate, TEMED and ddH₂O. The Stacking gels are comprised of 30% Acrylamide mix, 1.0M Tris (pH 6.8), 10% SDS, 10% Ammonium persulfate, TEMED and ddH₂O. The electrophoresis runs in 1X running buffer in 70V at room temperature for 30 min and continued in 120V at the room temperature for 35 min. After electrophoresis, the SDS-PAGE is transferred to the Polyscreen® PVDF transfer membrane (NEF1002001PK) at 120V and 4° C. for 100 min in 1X transfer buffer. The membrane is blocked in 5% skim milk with shaking at 70 rpm for 1 hour at room temperature. Then, the membrane is washed with PBST (Phosphate Buffered Saline with 0.1% Tween-20) for three-time with shaking 100 rpm for 5 min at room temperature. HRP-conjugated Mouse anti His-Tag mAB as the primary and secondary antibody is diluted by fresh 0.1% PBST and added 1000-fold to the membrane for 1 hour with shaking 70 rpm at room temperature. Then, the membrane is washed with 0.1% PBST for one time with shaking 100 rpm for 15 min and continued to second and third washed in same condition for each 5 min at room temperature. The last, membrane is imaged for detecting protein on a Multige121 imaging system.

Next, Step S103: cultivating the yeast transformant to obtain the PHB and performing analysis.

Screening PHB production from cultivation selected engineered Rhodotorula glutinis

Screening of PHB production is performed in shaking flasks. 1 ml of nine selected candidate strains of transformant Rhodotorula glutinis and wild-type (WT) are inoculated into a 250-ml flask containing the seed medium of a 50-mL working volume, pH 5.5. It is shaken at 150 rpm at 24° C. for 48h under aerobic conditions. Subsequently, 5 ml of seed medium (10%) are transferred into 250-ml flasks containing designated amounts of fermentation medium of a 50-ml working volume, pH 5.5, as described previously. In this part, glucose 30 g/L and galactose 30 g/L are used as carbon sources. It is cultured at 24° C. and shaken at 150 rpm under aerobic conditions. All shaking culture conditions are provided in triplicate to express the values as the mean±standard deviation.

Shaking flask cultivation of specific Rhodotorula glutinis strain #100-29

1 ml seed of specific Rhodotorula glutinis strain #100-29 is inoculated in a similar condition above. It is shaken at 150 rpm at 24° C. for 48h under aerobic conditions. Subsequently, it is cultured in a medium appropriate and condition for similar above. In this part, glucose 30 g/L, 45 g/L and 60 g/L and galactose, crude glycerol, oil each 30 g/L are used as carbon sources. The oil may be general oil such as animal oil, vegetable oil etc., or may be waste oil, the invention does not particularly limit the type of oil. All shaking culture conditions are provided in triplicate to express the values as the mean±standard deviation. In the NaCl experiment, 5 ml of seed culture was transferred into a 250-L flask shaking of a 50-L working volume of fermentation medium, pH 5.5. It was shaken at 150 rpm at 24° C. for 48h under aerobic conditions. In this part, carbon sources mixed in aerobic batch fermentation media using glucose (30 g/L) and crude glycerol in similar concentrations (30 g/L).

Aerobic batch bioreactor operation of specific Rhodotorula glutinis strain #100-29

First, 300 ml of seed culture is transferred into a 5-L batch bioreactor (model BTF-A, Biotop Ltd., Taiwan) of a 3-L working volume of fermentation medium (as described previously). The samples are carried out for interval times: 12h, 24 h, 36h, 48, 72h, 96h, 120h and 144h. The pH level is automatically maintained at 5.5 by automatically feeding 0.8M NaOH solution and 0.8M HCl into the medium. The fermentor is operated at 24° C. with dissolved oxygen controlled at a 30±10% saturation level. The agitation during the process is limited to a range of 200-400 rpm (300 rpm) with a 1.5 vvm aeration rate.

Biomass Measurement and Glucose Analysis

Biomass concentration is measured using an infrared balance (Denver Instrument, IR 35) and the procedure is prepared according to Yen H W, Hu C Y, Liang W S (2019) A cost efficient way to obtain lipid accumulation in the oleaginous yeast Rhodotorula glutinis using supplemental waste cooking oils (WCO). J Taiwan Inst Chem Eng 97:80-87. The concentration of carbon sources residues is quantified in the culture supernatant using an Ultimate 3000 HPLC refraction index (RI) detector (Agilent series 1100, Agilent Technologies, Santa Clara, Calif.) equipped with a Coregel 87H3 Column, ICE-99-9861, Serial No. 12528142. It was operated at 60° C. (glucose) and 55° C. (glycerol) and a flow rate of 0.6 mL/min of 0.01 N H₂SO₄ (glucose) and 0.008 N H₂SO₄ (glycerol).

Dried Cell Mass Determination

10 ml volume of culture samples are centrifuged at 7.000 rpm for 10 min and the pellets are washed once with distilled water and centrifuged in a similar condition. The pellet is kept in the freezer overnight. To lyophilize the biomass, the recovered cell pellet is immediately frozen in a freeze-dryer (CT-series, Panchum coin.) for 24h. The dry cell weight is determined and the pellet was kept at 4° C. for further analysis.

Measurement of PHB

PHB was analyzed as described in Karr D B, Waters J K, Emerich D W (1983) Analysis of poly-β-hydroxybutyrate in Rhizobium japonicum bacteroids by ion-exclusion high-pressure liquid chromatography and UV detection. Appl Environ Microbiol 46:1339-1344. Tyo K E, Zhou H, Stephanopoulos G N (2006) High-throughput screen for poly-3-hydroxybutyrate in Escherichia coli and synechocystis sp. strain PCC6803. Appl Environ Microbiol 72:3412-3417. Kocharin K, Chen Y, Siewers V, Nielsen J (2012) Engineering of acetyl-CoA metabolism for the improved production of polyhydroxybutyrate in Saccharomyces cerevisiae. AMB Express 2:1-11. Kocharin K, Siewers V, Nielsen J (2013) Improved polyhydroxybutyrate production by Saccharomyces cerevisiae through the use of the phosphoketolase pathway. Biotechnol Bioeng 110:2216-2224. Briefly, 40-50 mg of dried cells are weighed and boiled in 2 mL (95° C.) of concentrated sulfuric acid for 60 min. Samples are centrifuged (10 min, 7.000 rpm) to remove cell debris. Subsequently, the supernatant is diluted 20X of 0.014 N H₂SO₄, and filtered in membrane nylon B13NY045, Basefil Syringe Filter, 0.45 μm, area 1.09 cm², diameter 13 mm. The extract is analyzed in an HPLC-UV detector using a Coregel 87H3 Column, ICE-99-9861, Serial No. 13722909. Commercially standard PHB (Sigma-Aldrich, St. Louis, Mo.), processed in parallel with the samples, is used as a standard. The HPLC is operated at 60° C. and a flow rate of 0.6 ml/min of 0.014 N H₂SO₄.

Total Lipid Analysis

Extraction of lipids from wet biomass is based on a modification of the procedure used by Bligh, E. G. and Dyer W J (1959) Canadian Journal of Biochemistry and Physiology. Can J Biochem Physiol 37. Around 50-100 mg dry biomass from a crude powder is blended with 5 ml chloroform/methanol (2:1) and the mixture is agitated for 3 min at room temperature in an orbital shaker. The solvent phase is recovered by centrifugation at 7000 rpm for 10 min. The whole solvent is evaporated and dried under vacuum conditions.

Results

1. Expression of PhaA gene, PhaB gene, and PhaC gene in engineered Rhodotorula glutinis strains and accumulation of recombinant PHB

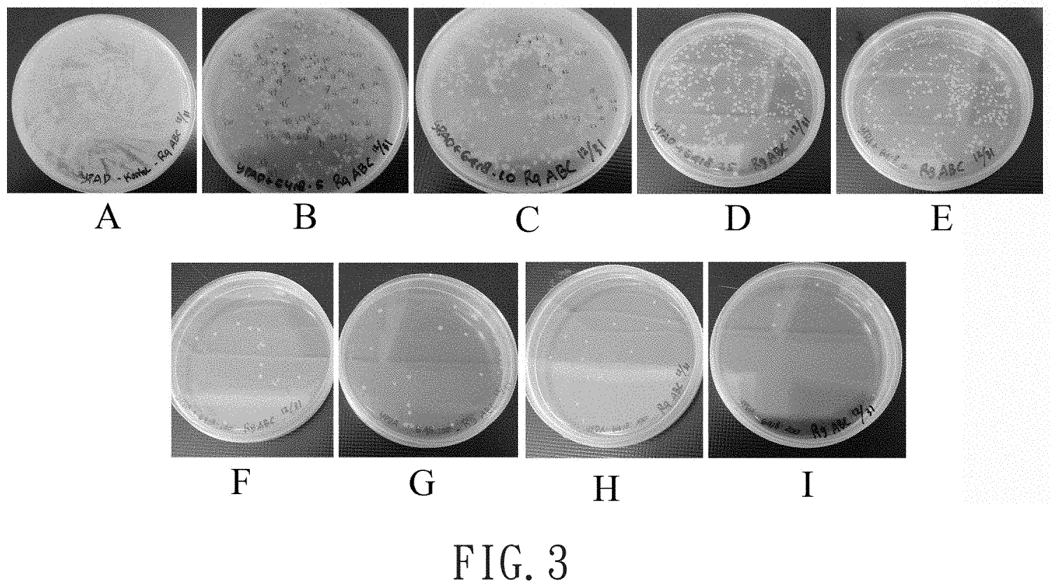

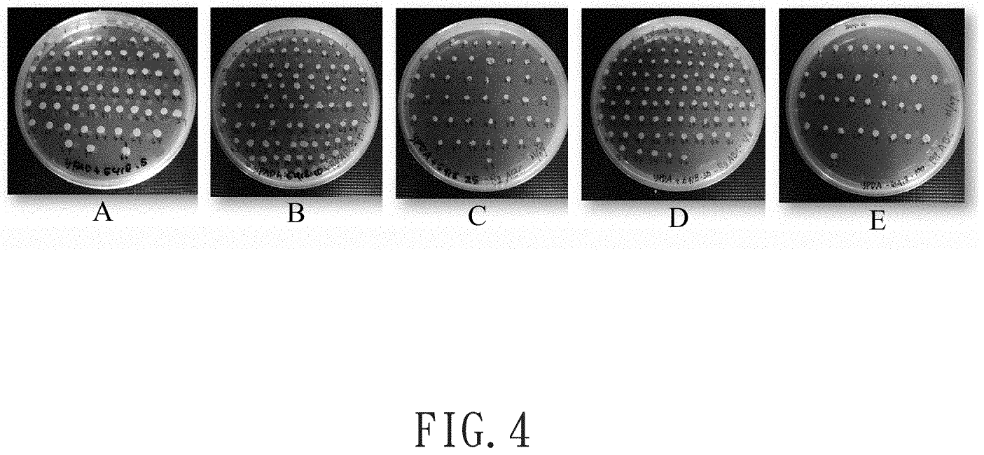

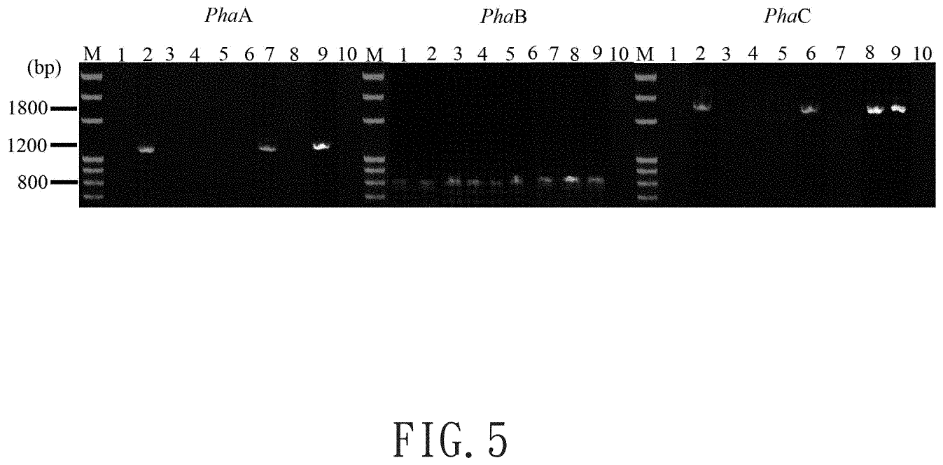

The transformation of three gene cassettes expression using strong promotor (pKLAC), PhaA gene, PhaB gene, and PhaC gene, are successfully integrated into the Rhodotorula glutinis genome, using modified lithium acetate competent cells in the electroporation method. The yeast transformants are screened using YP2D supplemented with G418 (5 μg/ml, 10 μg/ml, 25 μg/ml, 50 μg/ml, 100 μg/ml and 200 μg/ml) and the wild type without expression cassette is used as a control. FIG. 3 shows the viability of the antibiotic concentration G418 of the transformant Rhodotorula glutinis. A total of 384 transformants are randomly selected and sub-cultured to get stable transformants, as shown in FIG. 4. Subsequently, the yeast transformants are validated by PCR using the gene-specific primer pairs (SEQ ID NO: 4-9). In total, candidate colonies inserted transformant Rhodotorula glutinis for one, two or three genes are successfully screened as follows: 58 colonies have inserted PhaA-PhaB, 2 colonies have inserted PhaB-PhaC, 2 colonies PhaA-PhaB-PhaC, and 9 colonies have inserted PhaB and 1 colony have inserted PhaC, as shown in Table 1 below. Then, 10 selected colonies for the candidates the best strain which having a combination of three genes based on PCR confirmation are performed, as shown in FIG. 5.

TABLE 1
Determining the inhibitory viability of the antibiotic concentration
G418 of the transformant Rhodotorula glutinis

| The | Total | | | | | |
|---|---|---|---|---|---|---|
| concentration | of | Total of number colonies | | | | |
| of antibiotics | number | have been inserted genes | | | | |
| Geneticin | of | | | PhaA- | | |
| (G418) | selected | PhaA- | PhaB- | PhaB- | | |
| (μg/ml) | colonies | PhaB | PhaC | PhaC | PhaB | PhaC |
| 5 | 70 | 8 | 0 | 0 | 0 | 0 |
| 10 | 122 | 17 | 0 | 0 | 0 | 1 |
| 25 | 48 | 9 | 0 | 1 | 1 | 0 |
| 50 | 96 | 12 | 0 | 0 | 2 | 0 |
| 100 | 33 | 12 | 2 | 1 | 6 | 0 |
| 200 | 15 | 0 | 0 | 0 | 0 | 0 |

In the embodiment, candidates from three combination concentrations of antibiotics G418 in YPAD galactose (25 μg/ml, 50 μg/ml and 100 μg/ml) are screened. FIG. 5 illustrates the PCR confirmation from 10 selected candidates strain with inserted genes, in list: 25-39-PhaB; 25-44-PhaA-PhaB-PhaC; 50-39-PhaB; 50-87-PhaB; 100-12-PhaB; 100-13-PhaB-PhaC; 100-15-PhaA-PhaB; 100-16-PhaB-PhaC and 100-29-PhaA-PhaB-PhaC. 10 selected candidates are used to analyze overexpression PHB using western blot.

2. Characterization of overexpression PhaA gene, PhaB gene, and PhaC gene by Western Blot analysis

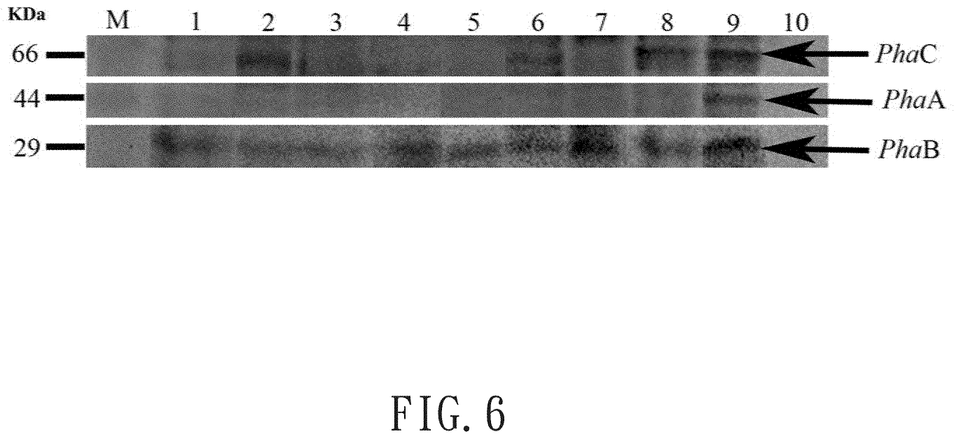

10 candidates recombinant Rhodotorula glutinis transformed with PhaA gene, PhaB gene and PhaC gene are tested for expression of the PHA synthase by Western blot analysis using an anti His-Tag, as shown in FIG. 6. Western blot is performed overexpression of PHB using three different sites of genes for PHB biosynthetic. FIG. 6 shows that the result of expression for each gene placed congruent with the PCR result (FIG. 5) and molecular mass of PhaA (˜66 KDa), PhaB (44 KDa) and PhaC (˜29 KDa), from the size of each inserted genes. Based on the analysis, it is strongly supported that strain #100-29 is the best candidate for further analyses because successfully expressed for three protein from PhaA gene, PhaB gene and PhaC gene of PHB biosynthesis.

The result shows a novel for Rhodotorula glutinis as oleaginous yeast successfully capable used to microbial cell factory to PHB production. Simultaneously, it could be the best and specific host to express three keys enzymes for the PHB production: acetyl-CoA C-acetyltransferase (encoded by PhaA), NADPH-dependent acetoacetyl-CoA reductase (encoded by PhaB), and PHA synthase (encoded by PhaC) in bacteria and some yeast. It is an important thing that the invention proves not only transformation in Saccharomyces cerevisiae capable to product the PHB, but Rhodotorula glutinis no doubtful more competent host because it also has many benefits for industrial manufacturing.

3. The screening of heterologous expression of PhaA gene, PhaB gene, and PhaC gene in nine candidates of selected transformants Rhodotorula glutinis

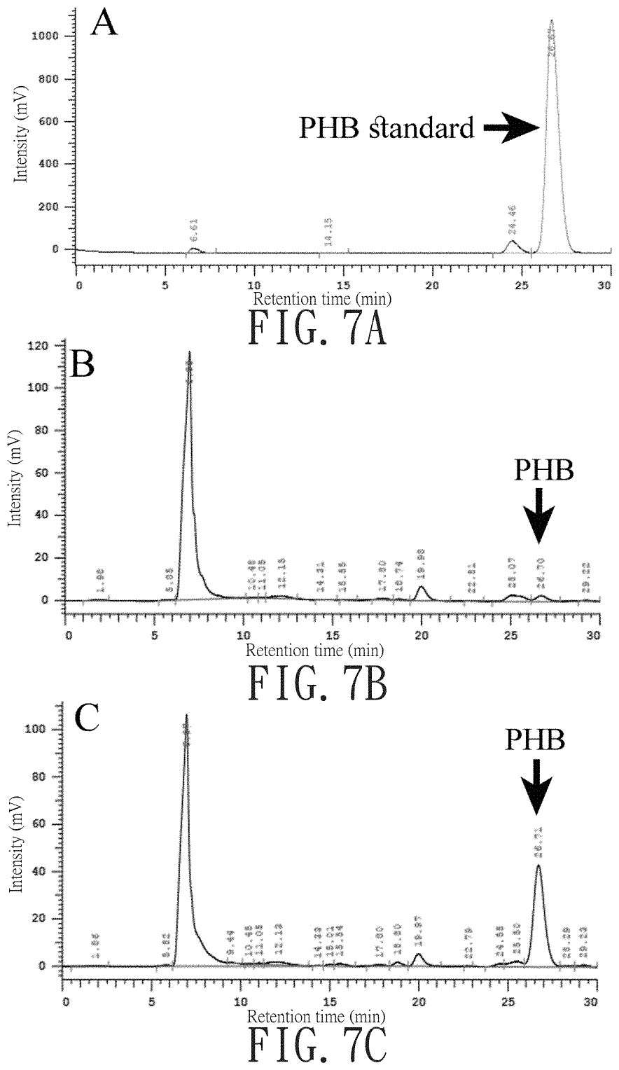

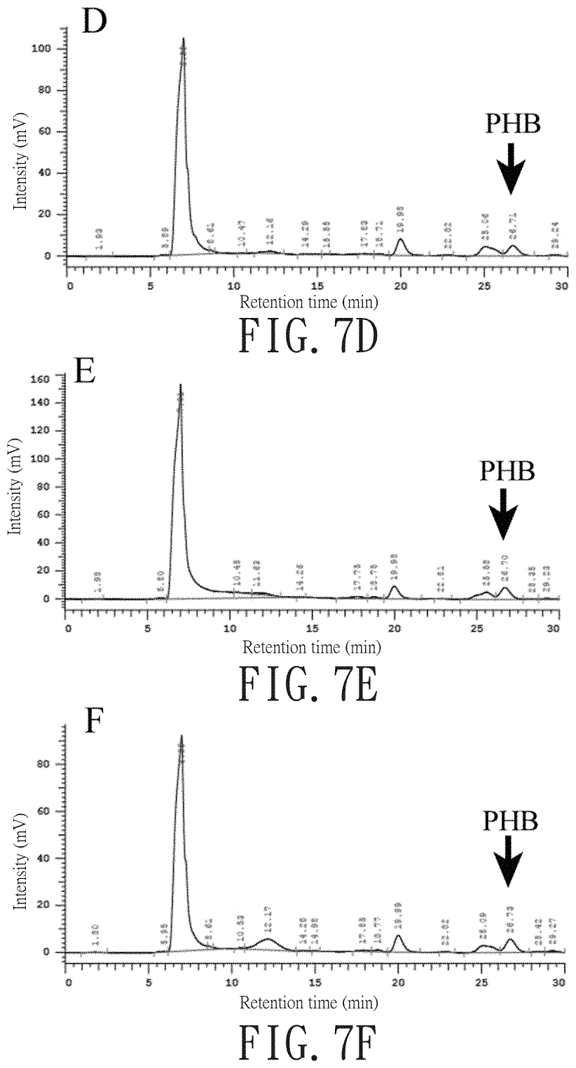

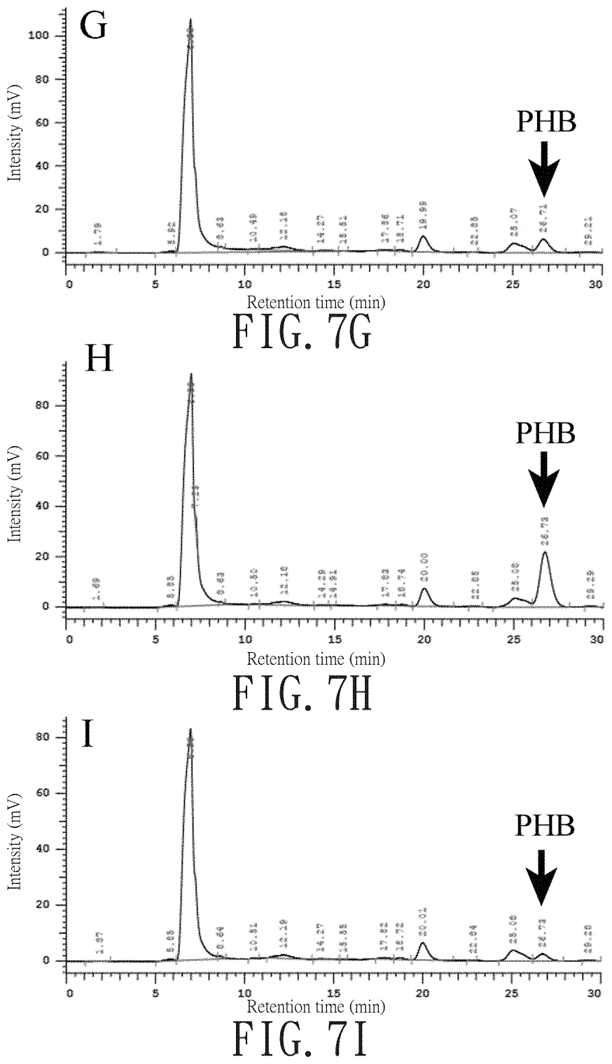

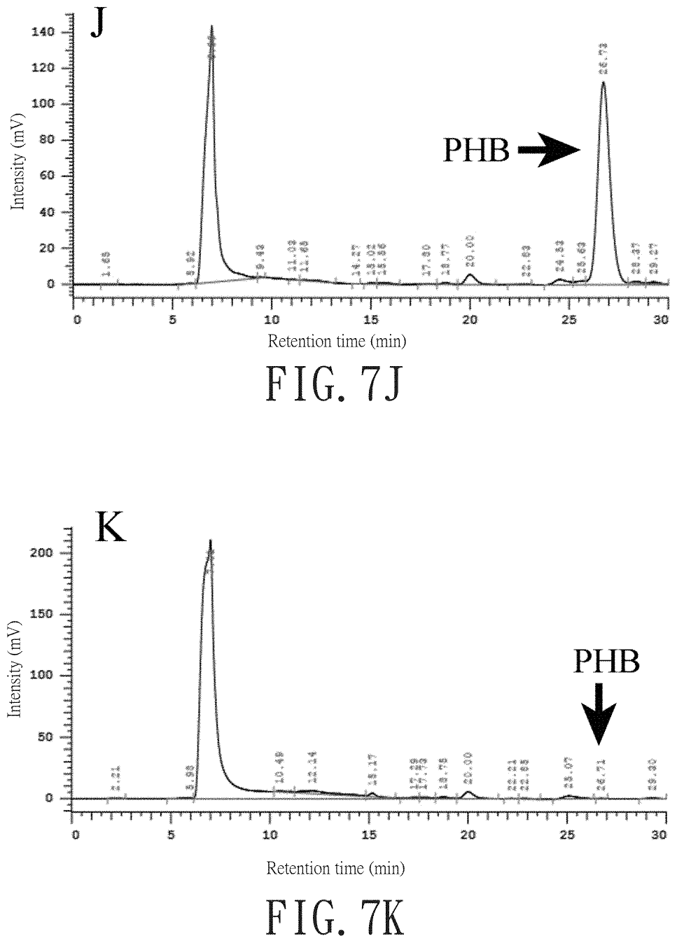

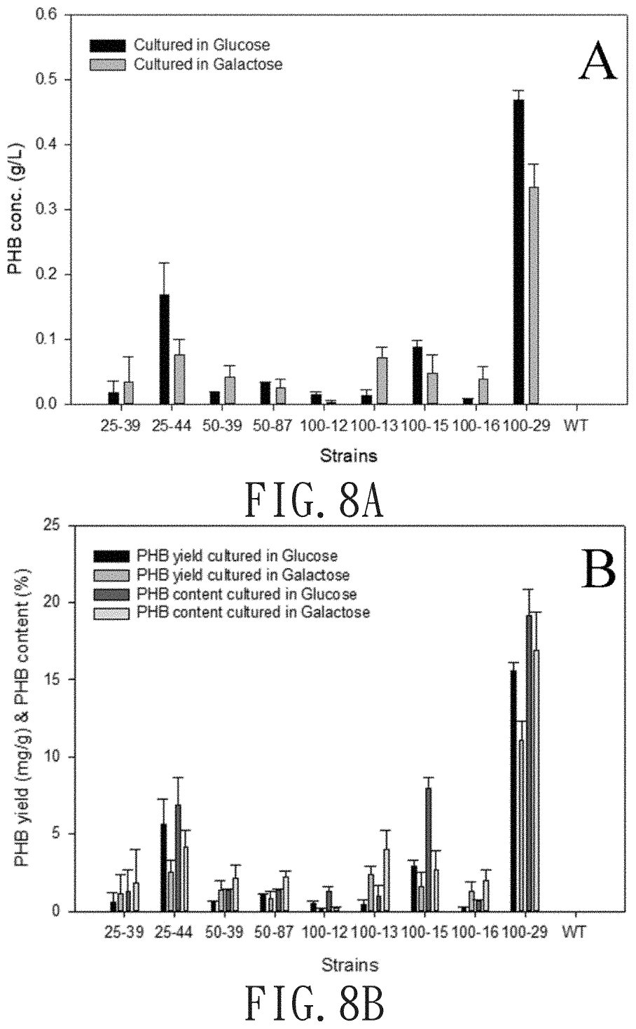

The investigation of heterologous expression of PHB has been successfully carried out on candidate transformant Rhodotorula glutinis using the western blot and HPLC method. FIGS. 7A-7K show different peak areas from nine candidates of transformants Rhodotorula glutinis, after being cultured in media using glucose 30 g/L as carbon sources. The PHB standard peak (FIG. 7A) shows in retention time of around 26 minutes, as well followed by nine candidate transformant Rhodotorula glutinis (FIG. 7B to FIG. 7K). FIG. 7J shows that strain 100-29 had higher pear areas and is supported with the result of PCR test and western blot analysis. Subsequently, to confirmation of the screening of PUB production from nine candidates transformants, the transformants are cultured again in galactose 30 g/L, the same condition with glucose 30 g/L. Both result cultivation from glucose 30 g/L and galactose 30 g/L, it is measured for PHB concentration, PHB yield and PHB content and the wild-type strain is employed as negative control, as shown in FIGS. 8A-8B.

In the embodiment, a method for PHB extraction and recovery from yeast cells using chemical disruption by purifying sulphuric acid (as described in the method) are demonstrated. The presence of PHB in intracellular of transformant Rhodotorula glutinis is confirmed by HPLC analysis, using commercial PHB standard control, as shown in FIGS. 7A-7K. The invention indicates that the HPLC method could be used for the screening and measure PHB concentration and the result may be further evaluated for PHB by fermentation techniques. The extraction PHB using sulphuric acid and quantified using HPLC in yeast and bacteria have been successfully established in the conventional technology field.

The result of PHB production was shown in FIGS. 8A-8B. Three combination concentrations of antibiotics G418 in YPAD galactose (25 μg/ml, 50 μg/ml and 100 μg/ml) are collected. Each combination is selected to get candidate strain of engineered Rhodotorula glutinis in list: 25-39; 25-44; 50-39; 50-87; 100-12; 100-13; 100-15; 100-16 and 100-29, congruent with the result of PCR test and western blot analysis. Based on FIG. 8 is known that from 9 candidates selected strain of engineered Rhodotorula glutinis, seed strain #100-29 performs the best strain for producing PHB. PHB production resulted in Rhodotorula glutinis strain #100-29 has highly PHB concentration, PHB yield and PHB content than other strains and wild-type. The highest of production PHB from strain #100-29 is demonstrated in both carbon sources (glucose and galactose). Therefore, strain #100-29 is chosen for further analyses. Glucose or galactose are added to the medium because to sustain exponential growth and PHB synthesis, as well as for shunt of PHB substrate by modification of glycolysis and others metabolic pathway. Likewise with cultivation has occurred in recombinant E. coli, some glucose and its derivates were added to optimize metabolic engineering and PHB biosynthesis.

4. Evaluation of growth and PHB production of selected engineered Rhodotorula glutinis strain #100-29 using shaking flask cultivation

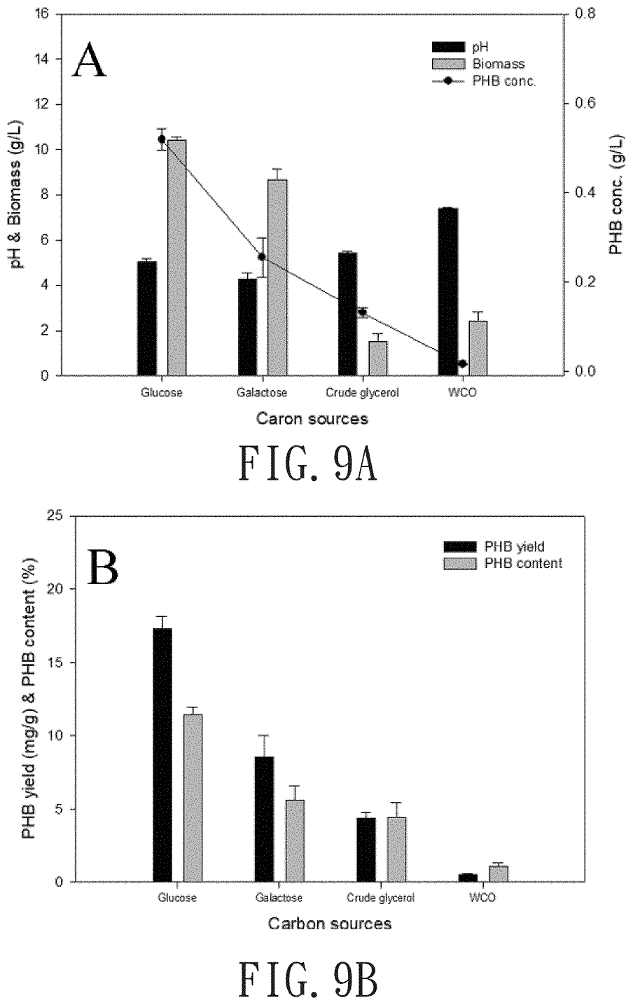

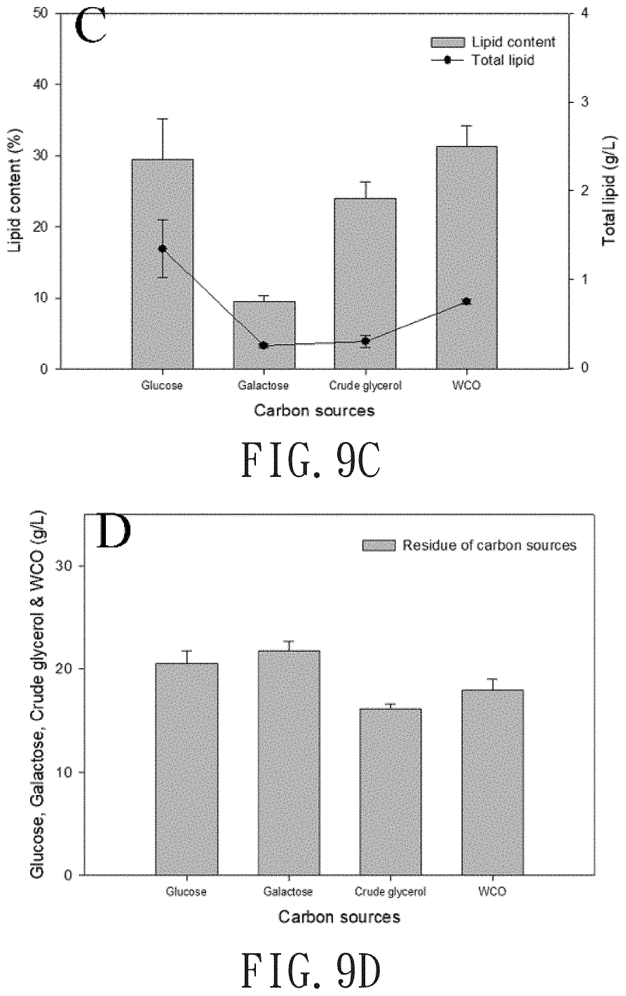

The selected engineered Rhodotorula glutinis strain #100-29 is evaluated of PHB production capacity in 250 ml shaken flasks of a 50-ml working volume for 48h. Firstly, Rhodotorula glutinis strain #100-29 is grown on media with different carbon sources in same concentration, such as glucose, galactose, crude glycerol and oil, each for 30 g/L (pH 5.5) to provide completely carbon and energy sources. Metabolite products are analyzed for biomass, PHB production, lipid and residue of carbon sources. The performance of engineered Rhodotorula glutinis strain #100-29 is displayed in FIGS. 9A-9D. Based on FIG. 9A shows that during fermentation time for 48h, pH was relatively stable from starting time cultivation (5.5) except for oil. Biomass and PHB concentration in glucose 30 g/L is higher than other carbon sources, up to 10 g/L and 0.52 g/L, respectively (FIG. 9A). In line with production PHB yield and PHB content, in glucose 30 g/L are higher than other carbon sources, up to 17 g/L and 11.4%, respectively (FIG. 9B). However, the production of lipid content in glucose and oil are higher than in other carbon sources, up to 30 g/L but for total lipid is higher in glucose, up to 18% (FIG. 9C). Subsequently, residuals of four kinds of carbon sources are shown an average of 33% less than initial carbon sources (30 g/L) for 48h (FIG. 9D).

The average residue of carbon sources from FIG. 9D is decreased to around 20 g/L from initial concentration (30 g/L), it means that in the culture time for 48h, in amount 10 g/L is used by microorganism to assimilate the carbon present in the glucose, galactose, crude glycerol and oil. Theoretically, carbon sources assimilation made NADPH in the pyruvate pathway from glucose, glycerol or ethanol. The result corresponds that 10 g/L is used to make adenosine triphosphate (ATP) will be transferred through the tricarboxylic acid cycle (TCA) used in the PHB production.

FIGS. 9A-9D show the performance engineered Rhodotorula glutinis strain #100-29 for different carbon sources (glucose, galactose, crude glycerol and oil) in PHB production and correlation with lipid production. The PHB production (FIG. 9A and FIG. 9B) is higher in glucose, probably because that glucose is the favorite carbon source of almost all microorganisms including yeast and is rapidly metabolized. It s very reasonable because that glucose s metabolized mainly through the glycolysis pathway. In contrast, lipid content in oil 30 g/L is higher than glucose but not for total lipid (FIG. 9C). It is probably because oil generally consists of 70% triacylglycerol and some oligomeric or polymeric triacylglycerols, diacylglycerols, monoacylglycerol, free fatty acids, aldehydes, and ketones. And all of them are the basic composition in metabolism lipids. This is also confirmed by the relatively highest consumption rate of glucose and oil (FIG. 9D). The reason for lower PHB productivity and lipid in crude glycerol in the shaking flask cultivation is because probably it does not have maximum enough time for fermentation.

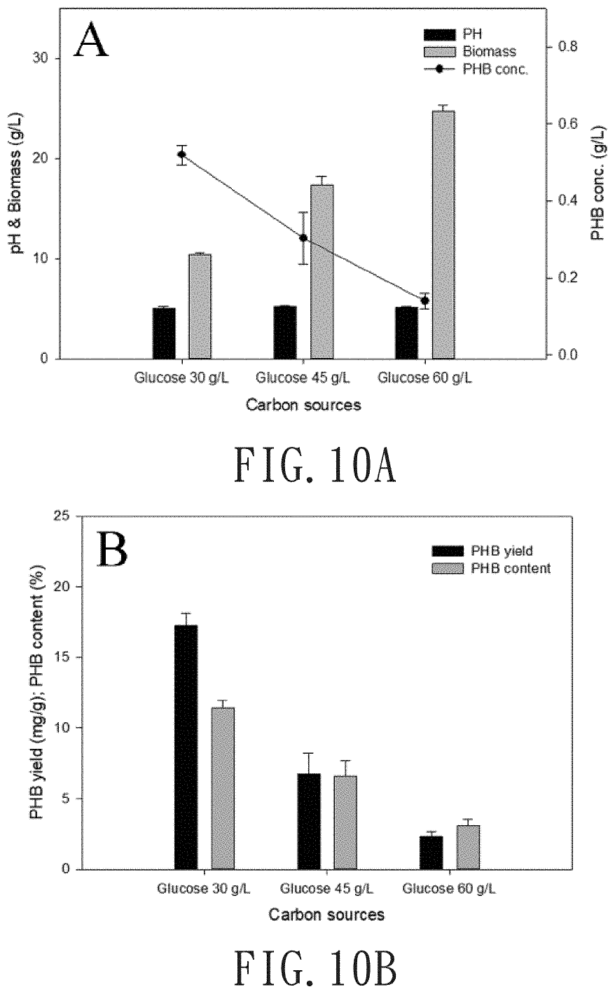

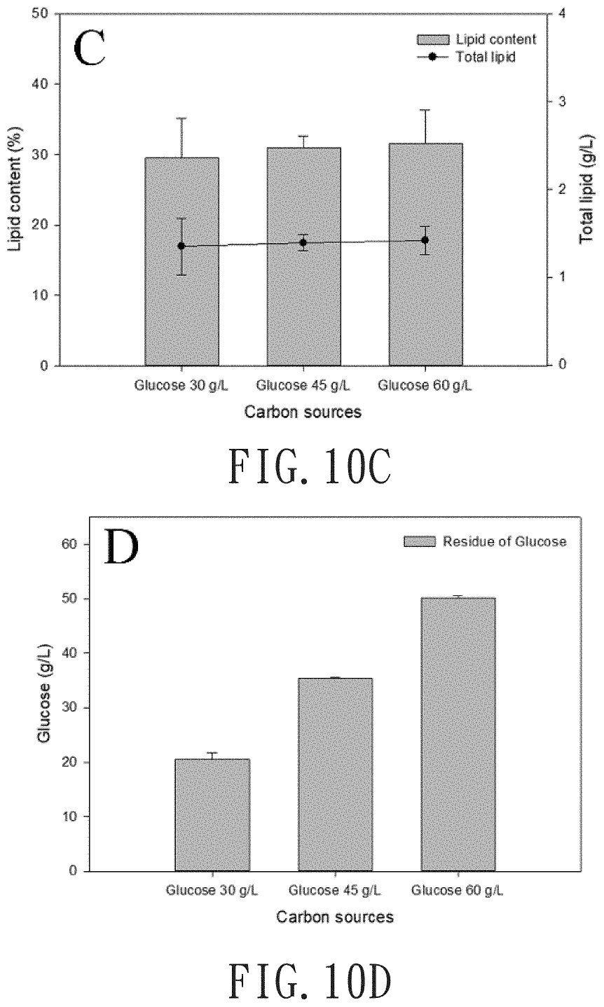

For evaluating the concentration of glucose, the selected engineered Rhodotorula glutinis strains #100-29 were cultured in 30 g/L, 45 g/L, and 60 g/L of glucose as shown in FIGS. 10A-10D. Increasing biomass production occurs during rising of glucose concentration from 30 g/L to 60 g/L, as shown in FIG. 10A and pH is similar in all concentrations. In contrast, the highest PHB production (PHB concentration, PHB yield and PHB content) is provided from selected engineered Rhodotorula glutinis strain #100-29 growth in glucose 30 g/L up to 0.52 g/L for PHB concentration, 17.3 mg/g for PHB yield and 11.4% for PHB content, as shown in FIG. 10A and FIG. 10B. On the other hand, FIG. 10C shows that selected engineered Rhodotorula glutinis strain #100-29 capable to produce lipid content relatively stable when growth in glucose (30 g/L, 45 g/L and 60 g/L). Subsequently, residual of three combination carbon sources are also shown an average of 15% less than initial carbon sources for 48h, as shown in FIG. 10D.

Different ranges of glucose (30 g/L, 45 g/L and 60 g/L) are utilized to optimize the best glucose range necessary for the maximum PHB production. Glucose-rich medium enhances the PHB production capability of yeast under nitrogen and phosphate limitation sources. Thus, glucose source as supplement carbon is added in the microorganism medium. FIG. 10A and FIG. 10B show that maximum PHB production occurred at 30% glucose concentration and at an incubation time of 48h. It means that in culture condition Glucose 30% is the best for PHB productivity (PHB concentration, PHB yield and PHB content rather than in glucose 45 g/L and 60 g/L (FIG. 10A and FIG. 10B). In fact, probably consider the limitation intolerance of higher concentration of carbon sources, because PHB will be produced in maximal production when microorganism growth under limiting conditions.

The ability to use a lower concentration of glucose as a carbon source is an important advantage, a cheap and abundant substrate. The maximum yield of PHB concerning the carbon source is very high in the organism. Nutrient limitation is necessary to trigger PHB accumulation, and generally, ammonia is used as the critical control factor for uncoupling the growth of cells and PHB production. A recombinant E. coli strain gave the maximum PHB content (about 60% PHB of DCW) at a specific combination of yeast extract and peptone. Similar results are obtained from the cultivation of Anaerobiospirillum succimproducens and Phaffia rhodozyma in the presence of yeast extract, and a combination of yeast extract and peptone. Furthermore, Table 3 shows that engineered Rhodotorula glutinis strain #100-29 had higher productivity PHB (PHB concentration, PHB yield and PHB content) when cultured in glucose and crude glycerol each 30 g/L rather than in transformant Saccharomyces cerevisiae in the previous study (references [1], [2], [4]-[6] and [8]). The maximum amount of PHB accumulated in engineered Saccharomyces cerevisiae strain C13ABYS86 6.7%, however in the embodiment of the invention are obtained up to 28.87% and 62% in glucose and crude glycerol, respectively.

TABLE 3
Comparison of PHB production from engineered yeast

| | | | | | | PHB | PHB | |
|---|---|---|---|---|---|---|---|---|
| | | | | | | yield/ | content | |
| | | | | | PHB | carbon | of | |
| Engineered | Carbon | | | Growth | conc. | sources | Biomass | |
| yeast | sources | Variation | | conditions | (g/L) | (mg/g)* | (%) | References |
| S. cerevisiae | Glucose | — | | Flask | — | — | 6.7 | [1] |
| strain | Galactose | | | | | | | |
| C13ABYS86 | | | | | | | | |
| Arxulaadenin | Glucose | — | | Flask | — | — | 0.11 | [2] |
| ivorans | | | | | | | | |
| S. cerevisiae | Glucose | — | | Bioreactor | — | 13.5 | — | [3] |
| strain RKS | Galactose | | | | | | | |
| S. cerevisiae | Glucose | — | | Flask | — | — | 2.93 | [4] |
| strain KYI | | | | | | | | |
| S. cerevisiae | | | | | | | | |
| strain | Glucose | — | | Flask | 0.050 | 0.13 | — | [5] |
| SCKK006 | | | | | | | | |
| S. cerevisiae | | | | | | | | |
| strain | Glucose | — | | Flask | 0.060 | 0.03 | — | [6] |
| SCKK032 | | | | | | | | |
| S. cerevisiae | | | | | | | | |
| strain | Glucose | — | | Bioreactor | 0.0054 | 5.59 | — | [7] |
| SCKK006 | | | | | | | | |
| S. cerevisiae | Xylose | | | Flask | 0.045 | 1.17 | 1.04 | [8] |
| strain TMB | | | | | | | | |
| 4443 | | | | | | | | |
| S. cerevisiae | | | | Bioreactor | 0.101 | 1.99 | 0.49 | [8] |
| strain TMB | | | | | | | | |
| 4444 | | | | | | | | |
| R. glutinis | Glucose, | Control | | Flask | 0.52 | 17.3 | 11.4 | The |
| strain 29 | 30 g/L | 1 | g/L | | 0.38 | 12.67 | 8.46 | invention |
| | | NaCl | | | | | | |
| | | 2 | g/L | | 0.41 | 13.57 | 9.11 | |
| | | NaCl | | | | | | |
| | | 3 | g/L | | 0.47 | 15.81 | 9.66 | |
| | | NaCl | | | | | | |
| | | 4 | g/L | | 0.27 | 8.94 | 5.71 | |
| | | NaCl | | | | | | |
| | | 5 | g/L | | 0.24 | 8.05 | 5.69 | |
| | | NaCl | | | | | | |
| | Glucose, | | | | 0.3 | 6.73 | 6.56 | |
| | 40 g/L | | | | | | | |
| | Glucose, | | | | 0.14 | 2.33 | 3.07 | |
| | 50 g/L | | | | | | | |
| | Glucose, | | | | 0.23 | 8.52 | 5.59 | |
| | 30 g/L | | | | | | | |
| | Crude | Control | | | 0.13 | 4.38 | 4.42 | |
| | glycerol, | 1 | g/L | | 0.75 | 25.00 | 58.07 | |
| | 30 g/L | NaCl | | | | | | |
| | | 2 | g/L | | 0.63 | 20.89 | 43.20 | |
| | | NaCl | | | | | | |
| | | 3 | g/L | | 0.60 | 20.12 | 41.27 | |
| | | NaCl | | | | | | |
| | | 4 | g/L | | 0.76 | 25.31 | 61.93 | |
| | | NaCl | | | | | | |
| | | 5 | g/L | | 0.44 | 14.80 | 33.62 | |
| | | NaCl | | | | | | |
| | Waste | | | | 0.002 | 0.54 | 1.11 | |
| | cooking | | | | | | | |
| | oil, | | | | | | | |
| | 30 g/L | | | | | | | |
| | Glucose, | | | | 1.3 | 43.35 | 28.87 | |
| | 30 g/L | | | | | | | |
| | Crude | 30 | g/L | Bioreactor | 2.87 | 95.53 | 62 | |
| | glycerol | 60 | g/L | | 3.4 | 92 | 72.3 | |
| | | 90 | g/L | | 0.12 | 1.33 | 23.89 | |

*PHB yield: the amount of total PHB from dried biomass in the initial carbon source.

5. Performance of selected engineered Rhodotorula glutinis strain #100-29 using aerobic batch bioreactor for PHB production

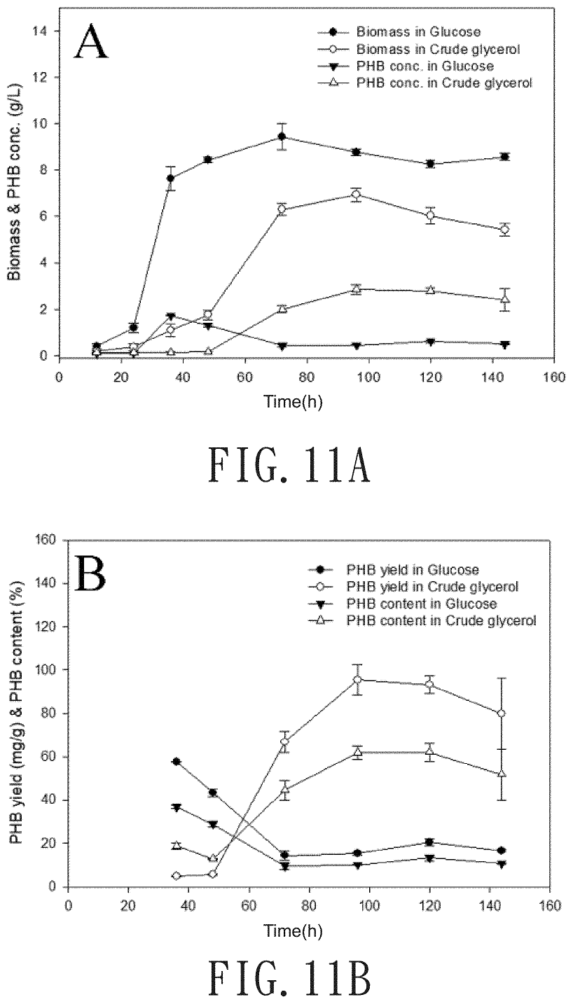

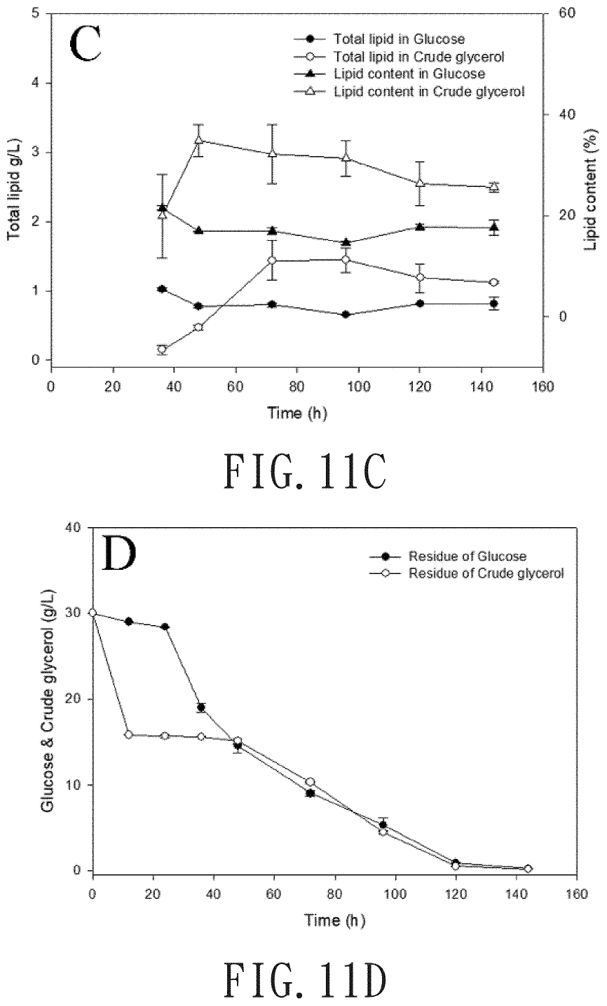

To further explore PHB production from engineered Rhodotorula glutinis strain #100-29, two independent fermentation aerobic batch bioreactors are carried out. The fermentation is performed in glucose 30 g/L and crude glycerol 30 g/L as carbon sources and pH-controlled (5.5) of 5 L bioreactor. Under the conditions, full glucose and glycerol consumption are observed within 12-144h, as shown in FIGS. 11A-11D. The production biomass during fermentation time is increased parallel in both conditions (glucose and crude glycerol), as shown in FIG. 11A. However, production biomass in glucose condition is tended higher rather than in crude glycerol, up to 9 g/L in maximum time. In contrast with PHB production (the highest PHB concentration, PHB yield and PHB content), the growth of engineered Rhodotorula glutinis strain #100-29 in the batch with crude glycerol has the highest rather than using glucose, up to 2.8 g/L for PHB concentration; 95.5 mg/g for PHB yield and 62% for PHB content, as shown in FIG. 11A and FIG. 11B and Table 3, in maximum time. Furthermore, the accumulation of lipid content in fermentation with crude glycerol has highest than in crude glycerol, as shown in FIG. 11C, up to 1.4 g/L (in maximum time) for total lipid and 30% for lipid content. On the other hand, the trend of residual glucose and glycerol consumption of both conditions has dramatically decreased during 12h-36h, starting from 30 g/L to almost 0.3 g/L until the end of fermentation time, as shown in FIG. 11D.

6. Performance of engineered Rhodotorula glutinis strain #100-29 using varieties concentrations of crude glycerol in the aerobic batch bioreactor for increasing PHB production

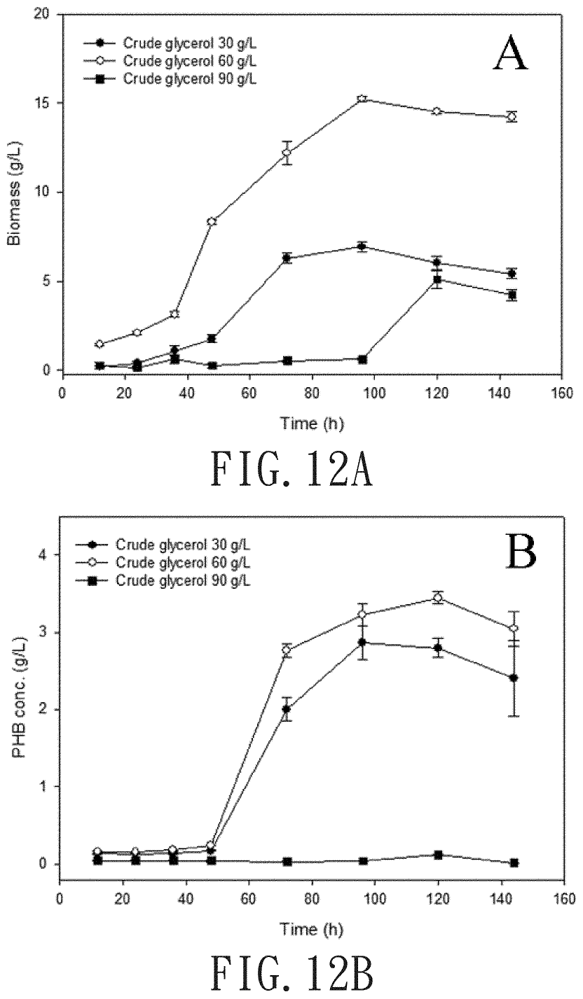

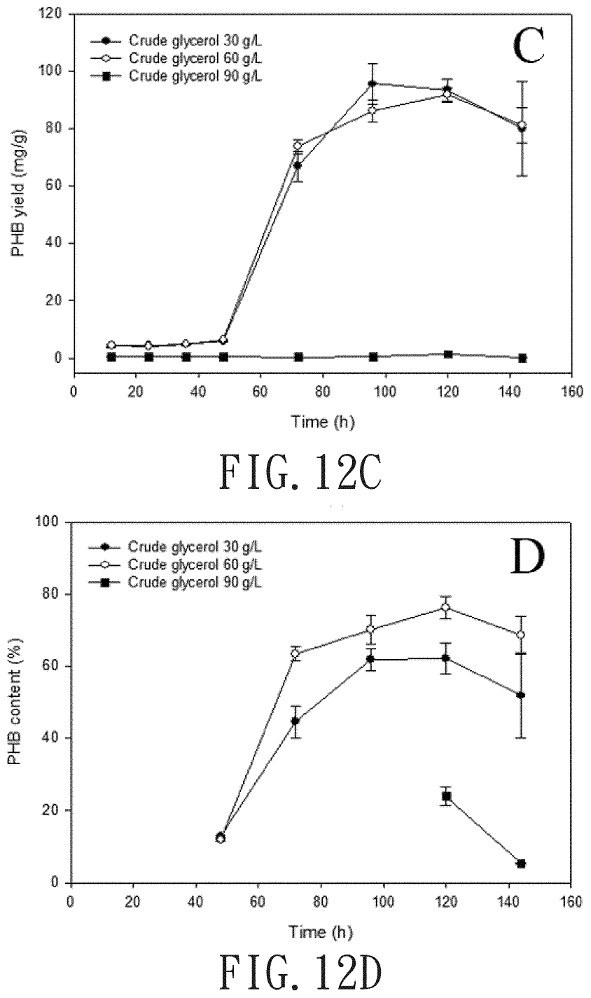

For evaluating the concentration of crude glycerol, the selected engineered R. glutinis strains #100-29 are cultured in 30 g/L, 60 g/L, and 90 g/L of crude glycerol. The production biomass during fermentation time is increased parallel in both conditions (crude glycerol 30 g/L and 60 g/L), as shown in FIG. 12A. However, production biomass in crude glycerol 60 g/L condition is tended higher rather than in 30 g/L or 90 g/L, up to 15.3 g/L in maximum time (96h). Similarly, the highest PHB production (PHB concentration, PHB yield and PHB content) is provided in crude glycerol 60 g/L up to 3.4 g/L for PHB concentration, 92 mg/g for PHB yield and 73.3% for PHB content, as shown in FIG. 12B to FIG. 12D. On the other hand, biomass and PHB production in crude glycerol 90 g/L is lower than two other combination concentrations. It means that in culture fermentation condition crude glycerol 60 g/L is the best for PHB productivity (PHB concentration, PHB yield and PHB content) rather than in glucose 30 g/L and 90 g/L. One plausible mechanism of decreasing biomass and PHB production in crude glycerol 90 g/L exhibited that it has strong inhibitions on substrate utilization and cell growth. Commonly, the increasing crude glycerol concentration also alternated microbial community and metabolic pathways. Moreover, in this pathway, microbial will respond to the limitation intolerance of higher concentration of carbon sources, because PHB will be produced in maximal production when microorganism growth under limiting conditions.

The PHB production in a 5 L aerobic batch bioreactor may improve the PHB production up to more than 3 times (as shown in Table 3) from shaking flask cultivation. Thus, significantly higher biomass could be obtained in a larger scale bioreactor. In the embodiment, PHB accumulation in crude glycerol when cultured in a batch bioreactor is higher than glucose in the same concentration (as shown in Table 3). Continuous culture offers many advantages for industrial production, provided that contamination is avoided and the stability of the strain is guaranteed. The advantages include simplicity of culture control, homogeneity of the production, and constancy of culture conditions.

The result in the invention represents that production PHB yield from engineered Rhodotorula glutinis strain #100-29 is higher than Rhodotorula glutinis var. glutinis 60 in the previous study, when it is cultured in a batch bioreactor using crude glycerol 30 g/L for the maximum time. Table 3 indicates that engineered Rhodotorula glutinis strain #100-29 has the highest PHB yield up to 43.35 mg/g in glucose and 95.53 mg/g in crude glycerol, rather than Saccharomyces cerevisiae strain RKS in glucose and galactose conditions. In addition, in the batch bioreactor, the higher concentration PHB and PHB yield are obtained from Rhodotorula glutinis strain #100-29 rather than Saccharomyces cerevisiae strain SCKK006 when it cultured with glucose. Moreover, Table 3 illustrates that in the batch bioreactor, the higher PHB content is discovered in glucose and crude glycerol (up to 28.87% and 62%, respectively) rather than Saccharomyces cerevisiae strain TMB 4444 when it cultured with xylose.

The utilization of crude glycerol for the production of PHA is an interesting alternative to support biodiesel production from fats and oils. Crude glycerol as a by-product of biodiesel production from rape is such a promising carbon source. This is an alternative and renewable energy source that will result in a surplus of glycerol. About 3.2 million tons of biodiesel per year were produced in Europe during 2005. From the invention, the utilization of crude glycerol as an ideal carbon source for industrial fermentation to produce PHB is successfully demonstrated.

As a co-product stream from biodiesel production, generally, composition minerals from crude glycerol are glycerol, free fatty acids (FFA), fatty acid methyl esters (FAME), and some traces of salts. And it will depend on the feedstock material, the transesterification process (catalytic way) and the recovery technology employed. Fatty acids and fatty acid methyl ester are the important components played role in acetyl-CoA. Acetyl-CoA is a central metabolite in carbon and energy metabolism which connects glycolysis, tricarboxylic acid cycle, β-oxidation, and de novo biosynthesis of fatty acids. Starting from acetyl-CoA, the biosynthesis of PHB, the simplest and most well-studied member of PHA family, is more suitable and faster catalyzed by three enzymes: β-ketothiolase, NADPH-dependent acetoacetyl-CoA reductase, and PHA synthase.

Conversion of the crude glycerol into PHAs is a promising route to offset the production cost of biodiesel and valorize the crude glycerol. The PHB content of engineered Rhodotorula glutinis strain #100-29 achieved a maximum 62% when cultured in crude glycerol (as shown in Table 3).

7. PHB production from engineered Rhodotorula glutinis strain #100-29 using varieties concentrations of NaCl in shaking flask

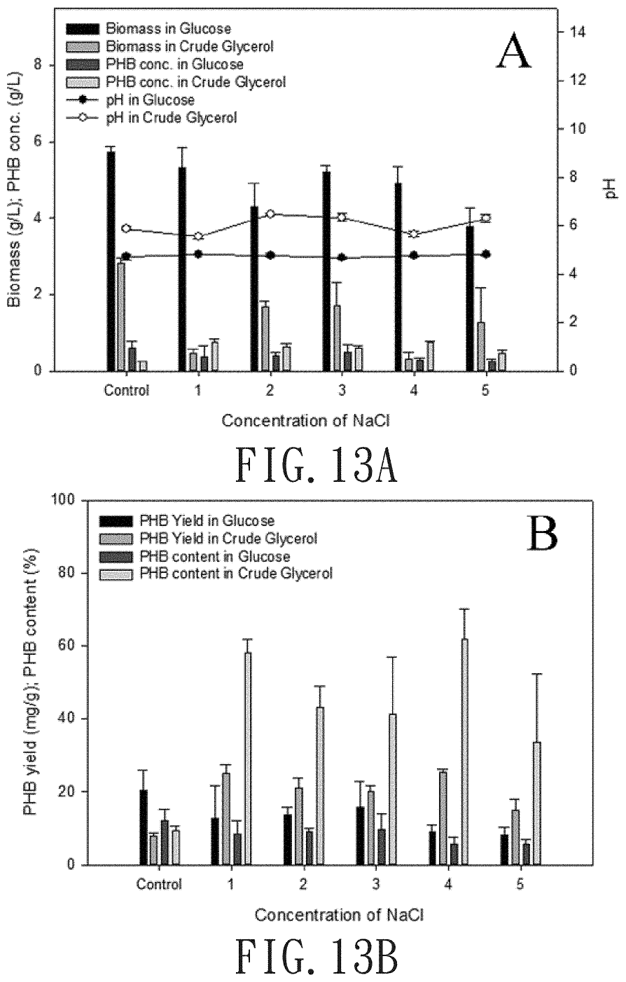

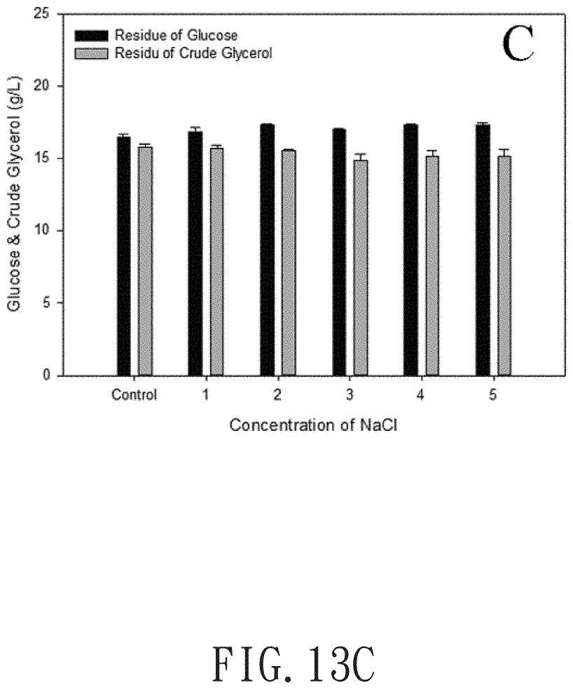

For evaluating the PHB production, the selected engineered R. glutinis strains #100-29 are cultured in control (0.1 g/L), 1, 2, 3, 4 and 5 g/L of NaCl (FIG. 13). The production PHB during fermentation time is increased parallel in both conditions (glucose and crude glycerol each 30 g/L). Average of biomass from all variation concentration NaCl in glucose condition is tended higher rather than in crude glycerol, up to 5.3 g/L (FIG. 13A). However, average PHB production (PHB concentration, PHB yield and PHB content) is higher in crude glycerol in 4 g/L NaCl rather than in glucose, up to 0.76 g/L for PHB concentration, 25.31 mg/g for PHB yield and 61.93% for PHB content (FIGS. 12B to 12C). Subsequently, residuals of carbon sources were shown an average of 50% less than initial carbon sources (30 g/L).

Also referring to Table 3, the data shows that in culture fermentation condition crude glycerol 30 g/L using 4 g/L NaCl is the optimum concentration of NaCl that can provide the adequate stress for the optimal PHB accumulation (PHB concentration, PHB yield and PHB content) rather than in another concentration. One plausible correlation is between osmotic stress and salt concentration. Therefore, NaCl addition may cause the yeast to eliminate water and cell content, to make more space for PHB granules and it become more packed under osmotic stress. As a result, the experiments show that the PHB production from engineered Rhodotorula glutinis may be carry out in brine or seawater. Compared to using pure water or distilled water in the experiment, seawater is cheap and easy to obtain, when the PHB production from engineered Rhodotorula glutinis is used in mass production, the advantage of seawater will be greater than pure water or distilled water.

In the embodiment of the invention, the enhancement of PHB production is improved by appropriated for genetic modifications in Rhodotorula glutinis. The engineered Rhodotorula glutinis from strain #100-29 as an eukaryotic organism has the capability to enhance PHB production (PHB concentration, PHB yield and PHB content). The improvement of PHB accumulation is obtained in flask shake culture with glucose, galactose, crude glycerol and oil as carbon sources, in which the lipid content in oil is higher than glucose. However, fermentation in the bioreactor is known more advantageous with crude glycerol as carbon sources. Crude glycerol is one of significant effort has been invested in the invention by using inexpensive carbon substrates and developing more efficient fermentation processes to reduce PHB production cost. Further, the implementation of biofuels along the biodegradable plastics could be a green outlet for petrochemical plastics and fuels, using common methods in the polymer industry. Rhodotorula glutinis strain #100-29 is a new PHB-producing strain adapted to convert crude glycerol. These microbial polyesters have high polymer accumulation rates which lead to future prospects concerning the optimization of cultivation parameters and the increase of cellular productivity. Furthermore, optimizations of the fermentation conditions are required to improve productivity. This result is applied to the next-generation industrial biotechnology (NGIB) offers extensive opportunities for competitive bioproduction.

In summary, since yeast has flexibility in physiology, is novel in biotechnology metabolism and larger than bacteria, it provides a cheaper and faster way to improve PHB production. In the method of the embodiment of the invention, an oleaginous yeast Rhodotorula glutinis is used in PHB production, which belongs to a type of yeast that has ability to provide lipids as a substrate for PHB synthesis in metabolic plasticity. It is demonstrated that an increasing of PHB production in oleaginous yeast by using optimation some carbon sources. Therefore, comparing with the method of using bacteria in the conventional technology, the oleaginous yeast of the embodiment of the invention provides a way of cheaper, faster and flexibility in biotechnology metabolism, and may further increase the PHB production.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

Reference:
1. Breuer U, Terentiev Y, Kunze G, Babel W (2002) Yeasts as producers of polyhydroxyalkanoates: Genetic engineering of Saccharomyces cerevisiae. Macromol Biosci 2:380-386. https://doi.org/10.1002/1616-5195(200211)2:8<380::AID-MAB1380>3.0. CO:2-X.
2. Terentiev Y, Breuer U, Babel W, Kunze G (2004) Non-conventional yeasts as producers of polyhydroxyalkanoates—Genetic engineering of Arxula adeninivorans. Appl Microbiol Biotechnol 64:376-381. https://doi.org/10.1007/s00253-003-1498-x
3. Carlson R, Srienc F (2006) Effects of recombinant precursor pathway variations on poly[(R)-3-hydroxybutyrate] synthesis in Saccharomyces cerevisiae. J Biotechnol 124:561-573. https://doi.org/10.1016/j.jbiotec.2006.01.035
4. Abd-El-haleem D, Amara A, Zaki S, et al (2007) Biosynthesis of biodegradable polyhydroxyalkanotes biopolymers in genetically modified yeasts. Int J Environ Sci Technol 4:513-520. https://doi.org/10.1007/BF03325988
5. Kocharin K, Chen Y, Siewers V, Nielsen J (2012) Engineering of acetyl-CoA metabolism for the improved production of polyhydroxybutyrate in Saccharomyces cerevisiae. AMB Express 2:1-11. https://doi.org/10.1186/2191-0855-52
6. Kocharin K, Siewers V, Nielsen J (2013) Improved polyhydroxybutyrate production by Saccharomyces cerevisiae through the use of the phosphoketolase pathway. Biotechnol Bioeng 110:2216-2224. https://doi.org/10.1002/bit.24888
7. Kocharin K, Nielsen J (2013) Specific growth rate and substrate dependent polyhydroxybutyrate production in Saccharomyces cerevisiae. AMB Express 3:1-6. https://doi.org/10.1186/2191-0855-3-18
8. Sandstrom A G, Muñoz de las Heras A, Portugal-Nunes D, Gorwa-Grauslund M F (2015) Engineering of Saccharomyces cerevisiae for the production of poly-3-d-hydroxybutyrate from xylose. AMB Express 5:1-9. https://doi.org/10.1186/s13568-015-0100-0

What is claimed is:

1. A method for biosynthesis polyhydroxybutyrate by a yeast transformant, comprising the following steps:
transforming a polyhydroxybutyrate biosynthesis related gene into an oleaginous yeast to obtain an yeast transformant;
screening the yeast transformant; and
cultivating the yeast transformant to obtain the polyhydroxybutyrate.

2. The method according to claim 1, wherein the oleaginous yeast is Rhodotorula glutinis.

3. The method according to claim 2, wherein a Rhodotorula glutinis strain is BCRC 22360.

4. The method according to claim 1, wherein the polyhydroxybutyrate biosynthesis related gene comprises at least one of PhaA gene, PhaB gene or PhaC gene.

5. The method according to claim 4, wherein the polyhydroxybutyrate biosynthesis related gene is PhaA gene, PhaB gene and PhaC gene.

6. The method according to claim 1, wherein the polyhydroxybutyrate biosynthesis related gene comprises at least one of a first gene having at least 90% sequence identity with a sequence of PhaA gene, a second gene having at least 90% sequence identity with a sequence of PhaB gene or a third gene having at least 90% sequence identity with a sequence of PhaC gene.

7. The method according to claim 1, wherein the method of transforming a polyhydroxybutyrate biosynthesis related gene into an oleaginous yeast to obtain the yeast transformant comprises inserting the polyhydroxybutyrate biosynthesis related gene into a linearized plasmid, and transforming the linearized plasmid into the oleaginous yeast.

8. The method according to claim 1, wherein a DNA of the yeast transformant has the polyhydroxybutyrate biosynthesis related gene.

9. The method according to claim 1, wherein the method of screening the yeast transformant comprises screening with a primer pair, one of the primer of the primer pair has a sequence as SEQ ID NO: 4, and the other primer has a sequence as SEQ ID NO: 5.

10. The method according to claim 1, wherein the method of screening the yeast transformant comprises screening with a primer pair, one of the primer of the primer pair has a sequence as SEQ ID NO: 6, and the other primer has a sequence as SEQ ID NO: 7.

11. The method according to claim 1, wherein the method of screening the yeast transformant comprises screening with a primer pair, one of the primer of the primer pair has a sequence as SEQ ID NO: 8, and the other primer has a sequence as SEQ ID NO: 9.

12. The method according to claim 1, wherein the method of cultivating the yeast transformant to obtain the polyhydroxybutyrate comprises cultivating under aerobic condition.

13. The method according to claim 1, wherein the method of cultivating the yeast transformant to obtain the polyhydroxybutyrate comprises cultivating the yeast transformant with oil.

14. The method according to claim 1, wherein a yield of polyhydroxybutyrate increase when cultivating the yeast transformant with glucose.

15. The method according to claim 1, wherein a production capacity of polyhydroxybutyrate per cell increase when cultivating the yeast transformant with glycerol or oil.

16. The method according to claim 1, wherein the method of cultivating the yeast transformant to obtain the polyhydroxybutyrate comprises cultivating the yeast transformant in seawater.
